(19) United States
(12) Patent Application Publication
(10) Pub. No.: US 2023/0190215 A1
(43) Pub. Date: Jun. 22, 2023

(54) CO-REGISTRATION OF INTRALUMINAL DATA TO NO CONTRAST X-RAY IMAGE FRAME AND ASSOCIATED SYSTEMS, DEVICE AND METHODS

(71) Applicant: PHILIPS IMAGE GUIDED THERAPY CORPORATION, SAN DIEGO, CA (US)

(72) Inventors: Ehud NACHTOMY, HERZLIYA (IL); Asher COHEN, SAN DIEGO, CA (US); Pei-Yin CHAO, EINDHOVEN (NL); Efrat PREISLER, HAIFA (IL); Michael ZARKH, QIRYAT ONO (IL)

(21) Appl. No.: 18/082,892

(22) Filed: Dec. 16, 2022

Related U.S. Application Data
(60) Provisional application No. 63/292,529, filed on Dec. 22, 2021.

(51) Int. Cl. A61B 6/00; A61B 34/20; A61B 6/12

(52) U.S. Cl. CPC A61B 6/5247; A61B 34/20; A61B 6/12; A61B 6/463; A61B 6/464; A61B 6/465; A61B 6/469; A61B 6/482; A61B 6/5217; A61B 6/5264; A61B 2034/2065; A61B 8/12

(57) ABSTRACT

A system includes a processor circuit that receives an extraluminal image obtained without contrast. The processor circuit receives multiple additional extraluminal images obtained without contrast as an intraluminal device is moved through a body lumen of a patient. The locations of the intraluminal device are tracked and used to form a curve. The curve is overlaid over one of the extraluminal images obtained without contrast. The curve and extraluminal image are displayed to a user and modified or confirmed. The intraluminal data points acquired by the intraluminal device are then co-registered to the extraluminal image. The extraluminal image and intraluminal data are displayed to the user.

### CROSS-REFERENCE TO RELATED APPLICATIONS

This application claims priority to and the benefit of U.S. Provisional Application No. 63/292,529, filed Dec. 22, 2021, which is incorporated by reference herein in its entirety.

### TECHNICAL FIELD

The present disclosure relates generally to coregistration of intraluminal and extraluminal data. In particular, intraluminal data is coregistered to an x-ray image obtained without contrast injection.

### BACKGROUND

Physicians use many different medical diagnostic systems and tools to monitor a patient's health and diagnose and treat medical conditions. Different modalities of medical diagnostic systems may provide a physician with different images, models, and/or data relating to internal structures within a patient. These modalities include invasive devices and systems, such as intravascular systems, and non-invasive devices and systems, such as external ultrasound systems or x-ray systems. Using multiple diagnostic systems to examine a patient's anatomy provides a physician with added insight into the condition of the patient.

In the field of intravascular imaging and physiology measurement, co-registration of data from invasive devices (e.g. intravascular ultrasound (IVUS) devices) with images collected non-invasively (e.g. via x-ray angiography and/or x-ray venography) is a powerful technique for improving the efficiency and accuracy of vascular catheterization procedures. Co-registration identifies the locations of intravascular data measurements along a blood vessel by mapping the data to an x-ray image of the vessel. A physician may then see on an angiography image exactly where along the vessel a measurement was made, rather than estimate the location.

Coregistration of intravascular data to locations along a blood vessel typically requires introduction of a contrast agent into the patient vasculature. The contrast agent makes otherwise non-radiopaque blood vessels appear in x-ray images. When displayed to a user, the locations of the intravascular data are displayed along the contrast-filled vessel in the x-ray image. Introducing contrast agent, however, can be time consuming and prone to error. Some patients may also not tolerate contrast agent well, which can cause discomfort for the patient.

### SUMMARY

Embodiments of the present disclosure are systems, devices, and methods for coregistering intraluminal data and/or annotations to locations along a vessel of an x-ray image obtained without contrast. In a no-contrast x-ray image, the vessel itself is not visible in the image. Aspects of the present disclosure advantageously allow a user to perform coregistration with a no-contrast x-ray image or a low-dose contrast x-ray image. This advantageously allows coregistration procedures to be performed for patients with Chronic Kidney Disease (CKD), or other sensitivities to x-ray contrast agent, without exposing them to contrast dyes. This also allows patients, particularly those with CKD, to be discharged after a coregistered intraluminal procedure the same day with less concern for the development of Contrast Induced Nephropathy (CIN). Same-day discharge is cost effective and safer for patients and has been shown to be safe even after the most complex interventions.

Aspects of the present invention may include zero contrast coregistration and/or optimizing co-registration workflow in interventional vascular procedures under x-ray which do not utilize contrast injection. Multiple zero-contrast x-ray images are obtained during an intravascular procedure. A radiopaque portion of an intravascular device is seen in each zero-contrast x-ray image. The positions of the device in each image form a pathway. The pathway is then processed and a motion-corrected centerline pathway is determined. This motion-corrected centerline pathway is overlaid over one of the zero-contrast x-ray images. The pathway is then displayed to a user. The user may edit the shape of the pathway and/or confirm that the shape of the pathway is correct. The positions at which intravascular data was collected may then be associated with locations along the pathway allowing a physician to see where intravascular data was obtained within an x-ray image.

In an exemplary aspect, a system is provided. The system includes a processor circuit configured for communication with an extraluminal imaging device and an intraluminal catheter or guidewire, wherein the processor circuit is configured to: receive a first extraluminal image obtained by the extraluminal imaging device; receive a plurality of second extraluminal images obtained by the extraluminal imaging device during movement of the intraluminal catheter or guidewire within a body lumen of a patient, wherein the plurality of second plurality of extraluminal images are obtained without a contrast agent within the body lumen; receive a plurality of intraluminal data points obtained by the intraluminal catheter or guidewire during the movement; determine, based on the plurality of second extraluminal images, a curve representative of at least one of a shape or a location of the body lumen; determine if the first extraluminal image was obtained without the contrast agent within the body lumen; in response to the determination that the first extraluminal image was obtained without the contrast agent within the body lumen: assign the curve to be a centerline of the body lumen in the first extraluminal image; co-register the plurality of intraluminal data points to positions along the curve; output, to a display in communication with the processor circuit, a first screen display comprising: the first extraluminal image; a visual representation of an intraluminal data point of the plurality of intraluminal data points; and a marking overlaid on the extraluminal image at a corresponding position of the intraluminal data point.

In one aspect, in response to the determination that the first extraluminal image was obtained without the contrast agent within the body lumen, the processor circuit is configured to: output, to the display, a second screen display comprising: the first extraluminal image; and the curve overlaid on the first extraluminal image. In one aspect, the second screen display comprises a plurality of user input options to at least one of accept the centerline, correct the centerline, or draw a new centerline. In one aspect, when a user input option to correct the centerline is selected, the processor circuit is configured to receive a user input to identify a region of the curve and select a new location within the first extraluminal image corresponding to a corrected location of the region. In one aspect, the processor is configured to perform the co-registration and output the first screen display only after receiving a user input via the plurality of user input options. In one aspect, the processor circuit is configured for communication with a touchscreen display, the processor circuit is configured to output the first screen display to the touchscreen display, and the processor circuit is configured to receive the user input from the touchscreen display. In one aspect, the extraluminal imaging device comprises an x-ray imaging device. In one aspect, the first extraluminal image is obtained with a first radiation dose and the plurality of second extraluminal images are obtained with a second radiation dose smaller than the first radiation dose. In one aspect, the processor circuit is configured to: receive a plurality of first extraluminal images obtained by the extraluminal imaging device; and select the first extraluminal image from among the plurality of first extraluminal images. In one aspect, the processor circuit is configured to determine if the first extraluminal image was obtained without the contrast agent automatically, without receiving a user input to identify that the first extraluminal image was obtained without the contrast agent. In one aspect, the plurality of second extraluminal images show a radiopaque portion of the intraluminal catheter or guidewire, and the processor circuit is configured to determine the curve based on the radiopaque portion shown in the plurality of second extraluminal images. In one aspect, the plurality of second extraluminal images are obtained during a plurality of anatomical cycles such that the intraluminal catheter or guidewire experiences periodic motion during the movement of the intraluminal catheter or guidewire through the body lumen, and to determine the curve, the processor circuit is configured to perform motion compensation. In one aspect, to perform the motion compensation, the processor circuit is further configured to locate the curve along a center of a shape generated by the movement of the intraluminal catheter or guidewire within the body lumen while the intraluminal catheter or guidewire experiences the periodic motion. In one aspect, the first extraluminal image is one of the plurality of second extraluminal images. In one aspect, the processor circuit is further configured to assign the curve to be a centerline of the body lumen in the first extraluminal image without identifying the body lumen in the first extraluminal image and without identifying the centerline in the first extraluminal image.

In an exemplary aspect, a method is provided. The method includes receiving, with a processor circuit in communication with an extraluminal imaging device, a first extraluminal image obtained by the extraluminal imaging device; receiving, with the processor circuit, a plurality of second extraluminal images obtained by the extraluminal imaging device during movement of the intraluminal catheter or guidewire within a body lumen of a patient, wherein the plurality of second plurality of extraluminal images are obtained without a contrast agent within the body lumen; receiving, with the processor circuit, a plurality of intraluminal data points obtained by an intraluminal catheter or guidewire during the movement through the body lumen, wherein the processor circuit is in communication with the intraluminal catheter or guidewire; determining, with the processor circuit, a curve representative of at least one of a shape or a location of the body lumen, based on the plurality of second extraluminal images; determining, with the processor circuit, if the first extraluminal image was obtained without the contrast agent within the body lumen; in response to the processor circuit determining that the first extraluminal image was obtained without the contrast agent within the body lumen (for example, the processor circuit, having made the determination that the extraluminal image was obtained without the contrast agent, performs the following steps): assigning, with the processor circuit, the curve to be a centerline of the body lumen in the first extraluminal image without identifying the body lumen in the first extraluminal image and without identifying the centerline in the first extraluminal image; co-registering, with the processor circuit, the plurality of intraluminal data points to positions along the curve; outputting, to a display in communication with the processor circuit, a first screen display comprising: the first extraluminal image; a visual representation of an intraluminal data point of the plurality of intraluminal data points; and a marking overlaid on the extraluminal image at a corresponding position of the intraluminal data point.

In an exemplary aspect, a system is provided. The system includes a processor circuit configured for communication with an extraluminal imaging device and an intraluminal catheter or guidewire, wherein the processor circuit is configured to: receive a first extraluminal image obtained by the extraluminal imaging device, wherein the first extraluminal image is obtained without contrast agent within the body lumen; receive a plurality of second extraluminal images obtained by the extraluminal imaging device during movement of the intraluminal catheter or guidewire within a body lumen of a patient, wherein the plurality of second plurality of extraluminal images are obtained without a contrast agent within the body lumen; receive a plurality of intraluminal data points obtained by the intraluminal catheter or guidewire during the movement; co-register the plurality of intraluminal data points to the first extraluminal image based on the plurality of second extraluminal images such that the co-registration is performed without an extraluminal image obtained with contrast agent within the body lumen; output, to a display in communication with the processor circuit, a first screen display comprising: the first extraluminal image; a visual representation of an intraluminal data point of the plurality of intraluminal data points; and a marking overlaid on the extraluminal image at a corresponding position of the intraluminal data point.

In an exemplary aspect, a system is provided. The system includes an intravascular imaging catheter; and a processor circuit configured for communication with an x-ray imaging device and the intravascular imaging device, wherein the processor circuit is configured to: receive a first x-ray image obtained by the x-ray imaging device; receive a plurality of second x-ray images obtained by the x-ray imaging device during movement of the intravascular imaging catheter within a blood vessel of a patient, wherein the plurality of second plurality of x-ray images are obtained without a contrast agent within the blood vessel; receive a plurality of intravascular images obtained by the intravascular imaging catheter during the movement; determine, based on the plurality of second x-ray images, a curve representative of at least one of a shape or a location of the blood vessel; determine if the first x-ray image was obtained without the contrast agent within the blood vessel; in response to the determination that the first x-ray image was obtained without the contrast agent within the blood vessel: assign the curve to be a centerline of the body lumen in the first x-ray image without identifying the blood vessel in the first x-ray image and without identifying the centerline in the first x-ray image; co-register the plurality of intravascular images to positions along the curve; output, to a display in communication with the processor circuit, a first screen display comprising: the first x-ray image; an intravascular image of the plurality of intravascular images; and a marking overlaid on the extraluminal image at a corresponding position of the intravascular image.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging and x-ray system, according to aspects of the present disclosure.

FIG. 2 is a diagrammatic top view of an ultrasound imaging assembly in a flat configuration, according to aspects of the present disclosure.

FIG. 3 is a diagrammatic perspective view of the ultrasound imaging assembly shown in FIG. 2 in a rolled configuration around a support member, according to aspects of the present disclosure.

FIG. 4 is a diagrammatic cross-sectional side view of the ultrasound imaging assembly shown in FIG. 3, according to aspects of the present disclosure.

FIG. 5 is a schematic diagram of a processor circuit, according to aspects of the present disclosure.

FIG. 6 is a diagrammatic view of an extraluminal image showing a pathway of an intraluminal device, according to aspects of the present disclosure.

FIG. 7 is a diagrammatic view of a relationship between extraluminal images and a set of locations, according to aspects of the present disclosure.

FIG. 8 is a diagrammatic view of a shape based on the pathway of an intraluminal device, according to aspects of the present disclosure.

FIG. 9 is a diagrammatic view of a footprint line of a shape based on the movement of an intraluminal device, according to aspects of the present disclosure.

FIG. 10 is a diagrammatic view of a relationship between intravascular ultrasound data, extraluminal images, and a footprint line of an intraluminal device, according to aspects of the present disclosure.

FIG. 11 is a diagrammatic view of a relationship between a footprint line and coregistered intraluminal data with a calculated centerline overlaid over an extraluminal image, according to aspects of the present disclosure.

FIG. 12 is a diagrammatic view of a graphical user interface, according to aspects of the present disclosure.

FIG. 13 is a diagrammatic view of a graphical user interface, according to aspects of the present disclosure.

FIG. 14 is a diagrammatic view of a graphical user interface, according to aspects of the present disclosure.

FIG. 15 is a diagrammatic view of a graphical user interface, according to aspects of the present disclosure.

FIG. 16 is a flow diagram of a method of coregistering intraluminal data to a no contrast x-ray image frame, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Aspects of the present disclosure invention seek to optimize workflow, user interface, and algorithmic aspects associated with co-registration of intraluminal data and extraluminal images which does not use contrast.

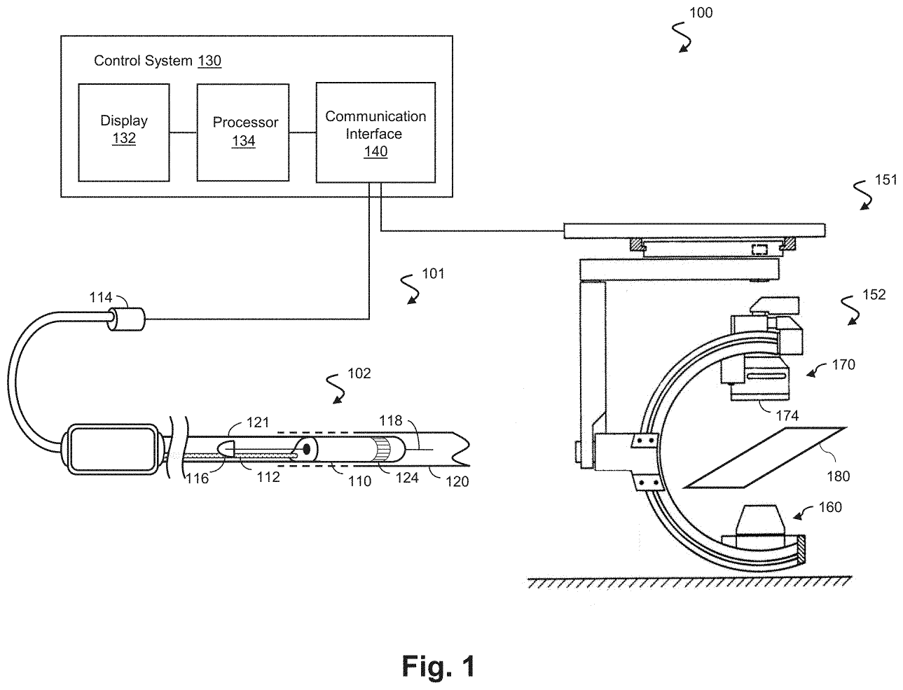

FIG. 1 is a schematic diagram of an intraluminal imaging and x-ray system 100, according to aspects of the present disclosure. In some embodiments, the intraluminal imaging and x-ray system 100 may include two separate systems or be a combination of two systems: an intraluminal sensing system 101 and an extraluminal imaging system 151. The intraluminal sensing system 101 obtains medical data about a patient's body while the intraluminal device 102 is positioned inside the patient's body. For example, the intraluminal sensing system 101 can control the intraluminal device 102 to obtain intraluminal images of the inside of the patient's body while the intraluminal device 102 is inside the patient's body. The extraluminal imaging system 151 obtains medical data about the patient's body while the extraluminal imaging device 152 is positioned outside the patient's body. For example, the extraluminal imaging system 151 can control extraluminal imaging device 152 to obtain extraluminal images of the inside of the patient's body while the extraluminal imaging device 152 is outside the patient's body.

The intraluminal imaging system 101 may be in communication with the extraluminal imaging system 151 through any suitable components. Such communication may be established through a wired cable, through a wireless signal, or by any other means. In addition, the intraluminal imaging system 101 may be in continuous communication with the x-ray system 151 or may be in intermittent communication. For example, the two systems may be brought into temporary communication via a wired cable, or brought into communication via a wireless communication, or through any other suitable means at some point before, after, or during an examination. In addition, the intraluminal system 101 may receive data such as x-ray images, annotated x-ray images, metrics calculated with the x-ray imaging system 151, information regarding dates and times of examinations, types and/or severity of patient conditions or diagnoses, patient history or other patient information, or any suitable data or information from the x-ray imaging system 151. The x-ray imaging system 151 may also receive any of these data from the intraluminal imaging system 101. In some embodiments, and as shown in FIG. 1, the intraluminal imaging system 101 and the x-ray imaging system 151 may be in communication with the same control system 130. In this embodiment, both systems may be in communication with the same display 132, processor 134, and communication interface 140 shown as well as in communication with any other components implemented within the control system 130.

In some embodiments, the system 100 may not include a control system 130 in communication with the intraluminal imaging system 101 and the x-ray imaging system 151. Instead, the system 100 may include two separate control systems. For example, one control system may be in communication with or be a part of the intraluminal imaging system 101 and an additional separate control system may be in communication with or be a part of the x-ray imaging system 151. In this embodiment, the separate control systems of both the intraluminal imaging system 101 and the x-ray imaging system 151 may be similar to the control system 130. For example, each control system may include various components or systems such as a communication interface, processor, and/or a display. In this embodiment, the control system of the intraluminal imaging system 101 may perform any or all of the coregistration steps described in the present disclosure. Alternatively, the control system of the x-ray imaging system 151 may perform the coregistration steps described.

The intraluminal imaging system 101 can be an ultrasound imaging system. In some instances, the intraluminal imaging system 101 can be an intravascular ultrasound (IVUS) imaging system. The intraluminal imaging system 101 may include an intraluminal imaging device 102, such as a catheter, guide wire, or guide catheter, in communication with the control system 130. The control system 130 may include a display 132, a processor 134, and a communication interface 140 among other components. The intraluminal imaging device 102 can be an ultrasound imaging device. In some instances, the device 102 can be an IVUS imaging device, such as a solid-state IVUS device. In some aspects, a user input device and the display 132 can be integrated into one housing in some instances, or may be separate devices.

At a high level, the IVUS device 102 emits ultrasonic energy from a transducer array 124 included in a scanner assembly, also referred to as an IVUS imaging assembly, mounted near a distal end of the catheter device. The ultrasonic energy is reflected by tissue structures in the surrounding medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The communication interface 140 transfers the received echo signals to the processor 134 of the control system 130 where the ultrasound image (including flow information in some embodiments) is reconstructed and displayed on the display 132. The control system 130, including the processor 134, can be operable to facilitate the features of the IVUS imaging system 101 described herein. For example, the processor 134 can execute computer readable instructions stored on the non-transitory tangible computer readable medium.

The communication interface 140 facilitates communication of signals between the control system 130 and the scanner assembly 110 included in the IVUS device 102. This communication includes the steps of: (1) providing commands to integrated circuit controller chip(s) included in the scanner assembly 110 to select the particular transducer array element(s), or acoustic element(s), to be used for transmit and receive, (2) providing the transmit trigger signals to the integrated circuit controller chip(s) included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) of the scanner assembly 110. In some embodiments, the communication interface 140 performs preliminary processing of the echo data prior to relaying the data to the processor 134. In examples of such embodiments, the communication interface 140 performs amplification, filtering, and/or aggregating of the data. In an embodiment, the communication interface 140 also supplies high- and low-voltage DC power to support operation of the device 102 including circuitry within the scanner assembly 110.

The processor 134 receives the echo data from the scanner assembly 110 by way of the communication interface 140 and processes the data to reconstruct an image of the tissue structures in the medium surrounding the scanner assembly 110. The processor 134 outputs image data such that an image of the lumen 120, such as a cross-sectional image of the vessel 120, is displayed on the display 132. The lumen 120 may represent fluid filled or surrounded structures, both natural and man-made. The lumen 120 may be within a body of a patient. The lumen 120 may be a blood vessel, such as an artery or a vein of a patient's vascular system, including cardiac vasculature, peripheral vasculature, neural vasculature, renal vasculature, and/or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter, Visions PV 0.014P RX catheter, Visions PV 0.018 catheter, Visions PV 0.035, and Pioneer Plus catheter, each of which are available from Koninklijke Philips N.V, and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors. It is understood that any suitable gauge wire can be used for the conductors. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a patient interface module (PIM) connector 114 at a proximal end of the device 102. The PIM connector 114 electrically couples the transmission line bundle 112 to the communication interface 140 and physically couples the IVUS device 102 to the communication interface 140. In some embodiments, the communication interface 140 may be a PIM. In an embodiment, the IVUS device 102 further includes a guide wire exit port 116. Accordingly, in some instances the IVUS device 102 is a rapid-exchange catheter. The guide wire exit port 116 allows a guide wire 118 to be inserted towards the distal end to direct the device 102 through the vessel 120.

In some embodiments, the intraluminal imaging device 102 may acquire intravascular images of any suitable imaging modality, including optical coherence tomography (OCT) and intravascular photoacoustic (IVPA).

In some embodiments, the intraluminal device 102 is a pressure sensing device (e.g., pressure-sensing guidewire) that obtains intraluminal (e.g., intravascular) pressure data, and the intraluminal system 101 is an intravascular pressure sensing system that determines pressure ratios based on the pressure data, such as fractional flow reserve (FFR), instantaneous wave-free ratio (iFR), and/or other suitable ratio between distal pressure and proximal/aortic pressure (Pd/Pa). In some embodiments, the intraluminal device 102 is a flow sensing device (e.g., flow-sensing guidewire) that obtains intraluminal (e.g., intravascular) flow data, and the intraluminal system 101 is an intravascular flow sensing system that determines flow-related values based on the pressure data, such as coronary flow reserve (CFR), flow velocity, flow volume, etc.

The x-ray imaging system 151 may include an x-ray imaging apparatus or device 152 configured to perform x-ray imaging, angiography, fluoroscopy, radiography, venography, among other imaging techniques. The x-ray imaging system 151 can generate a single x-ray image (e.g., an angiogram or venogram) or multiple (e.g., two or more) x-ray images (e.g., a video and/or fluoroscopic image stream) based on x-ray image data collected by the x-ray device 152. The x-ray imaging device 152 may be of any suitable type, for example, it may be a stationary x-ray system such as a fixed c-arm x-ray device, a mobile c-arm x-ray device, a straight arm x-ray device, or a u-arm device. The x-ray imaging device 152 may additionally be any suitable mobile device. The x-ray imaging device 152 may also be in communication with the control system 130. In some embodiments, the x-ray system 151 may include a digital radiography device or any other suitable device.

The x-ray device 152 as shown in FIG. 1 includes an x-ray source 160 and an x-ray detector 170 including an input screen 174. The x-ray source 160 and the detector 170 may be mounted at a mutual distance. Positioned between the x-ray source 160 and the x-ray detector 170 may be an anatomy of a patient or object 180. For example, the anatomy of the patient (including the vessel 120) can be positioned between the x-ray source 160 and the x-ray detector 170.

The x-ray source 160 may include an x-ray tube adapted to generate x-rays. Some aspects of the x-ray source 160 may include one or more vacuum tubes including a cathode in connection with a negative lead of a high-voltage power source and an anode in connection with a positive lead of the same power source. The cathode of the x-ray source 160 may additionally include a filament. The filament may be of any suitable type or constructed of any suitable material, including tungsten or rhenium tungsten, and may be positioned within a recessed region of the cathode. One function of the cathode may be to expel electrons from the high voltage power source and focus them into a well-defined beam aimed at the anode. The anode may also be constructed of any suitable material and may be configured to create x-radiation from the emitted electrons of the cathode. In addition, the anode may dissipate heat created in the process of generating x-radiation. The anode may be shaped as a beveled disk and, in some embodiments, may be rotated via an electric motor. The cathode and anode of the x-ray source 160 may be housed in an airtight enclosure, sometimes referred to as an envelope.

In some embodiments, the x-ray source 160 may include a radiation object focus which influences the visibility of an image. The radiation object focus may be selected by a user of the system 100 or by a manufacture of the system 100 based on characteristics such as blurring, visibility, heat-dissipating capacity, or other characteristics. In some embodiments, an operator or user of the system 100 may switch between different provided radiation object foci in a point-of-care setting.

The detector 170 may be configured to acquire x-ray images and may include the input screen 174. The input screen 174 may include one or more intensifying screens configured to absorb x-ray energy and convert the energy to light. The light may in turn expose a film. The input screen 174 may be used to convert x-ray energy to light in embodiments in which the film may be more sensitive to light than x-radiation. Different types of intensifying screens within the image intensifier may be selected depending on the region of a patient to be imaged, requirements for image detail and/or patient exposure, or any other factors. Intensifying screens may be constructed of any suitable materials, including barium lead sulfate, barium strontium sulfate, barium fluorochloride, yttrium oxysulfide, or any other suitable material. The input screen 374 may be a fluorescent screen or a film positioned directly adjacent to a fluorescent screen. In some embodiments, the input screen 374 may also include a protective screen to shield circuitry or components within the detector 370 from the surrounding environment. In some embodiments, the x-ray detector 170 may include a flat panel detector (FPD). The detector 170 may be an indirect conversion FPD or a direct conversion FPD. The detector 170 may also include charge-coupled devices (CCDs). The x-ray detector 370 may additionally be referred to as an x-ray sensor.

The object 180 may be any suitable object to be imaged. In an exemplary embodiment, the object may be the anatomy of a patient. More specifically, the anatomy to be imaged may include chest, abdomen, the pelvic region, neck, legs, head, feet, a region with cardiac vasculature, or a region containing the peripheral vasculature of a patient and may include various anatomical structures such as, but not limited to, organs, tissue, blood vessels and blood, gases, or any other anatomical structures or objects. In other embodiments, the object may be or include man-made structures.

In some embodiments, the x-ray imaging system 151 may be configured to obtain x-ray images without contrast. In some embodiments, the x-ray imaging system 151 may be configured to obtain x-ray images with contrast (e.g., angiogram or venogram). In such embodiments, a contrast agent or x-ray dye may be introduced to a patient's anatomy before imaging. The contrast agent may also be referred to as a radiocontrast agent, contrast material, contrast dye, or contrast media. The contrast dye may be of any suitable material, chemical, or compound and may be a liquid, powder, paste, tablet, or of any other suitable form. For example, the contrast dye may be iodine-based compounds, barium sulfate compounds, gadolinium-based compounds, or any other suitable compounds. The contrast agent may be used to enhance the visibility of internal fluids or structures within a patient's anatomy. The contrast agent may absorb external x-rays, resulting in decreased exposure on the x-ray detector 170.

In some embodiments, the extraluminal imaging system 151 could be any suitable extraluminal imaging device, such as computed tomography (CT) or magnetic resonance imaging (MRI).

When the control system 130 is in communication with the x-ray system 151, the communication interface 140 facilitates communication of signals between the control system 130 and the x-ray device 152. This communication includes providing control commands to the x-ray source 160 and/or the x-ray detector 170 of the x-ray device 152 and receiving data from the x-ray device 152. In some embodiments, the communication interface 140 performs preliminary processing of the x-ray data prior to relaying the data to the processor 134. In examples of such embodiments, the communication interface 140 may perform amplification, filtering, and/or aggregating of the data. In an embodiment, the communication interface 140 also supplies high- and low-voltage DC power to support operation of the device 152 including circuitry within the device.

The processor 134 receives the x-ray data from the x-ray device 152 by way of the communication interface 140 and processes the data to reconstruct an image of the anatomy being imaged. The processor 134 outputs image data such that an image is displayed on the display 132. In an embodiment in which the contrast agent is introduced to the anatomy of a patient and a venogram is to be generated, the particular areas of interest to be imaged may be one or more blood vessels or other section or part of the human vasculature. The contrast agent may identify fluid filled structures, both natural and/or man-made, such as arteries or veins of a patient's vascular system, including cardiac vasculature, peripheral vasculature, neural vasculature, renal vasculature, and/or any other suitable lumen inside the body. For example, the x-ray device 152 may be used to examine any number of anatomical locations and tissue types, including without limitation all the organs, fluids, or other structures or parts of an anatomy previously mentioned. In addition to natural structures, the x-ray device 152 may be used to examine man-made structures such as any of the previously mentioned structures.

The processor 134 may be configured to receive an x-ray image that was stored by the x-ray imaging device 152 during a clinical procedure. The images may be further enhanced by other information such as patient history, patient record, IVUS imaging, pre-operative ultrasound imaging, pre-operative CT, or any other suitable data.

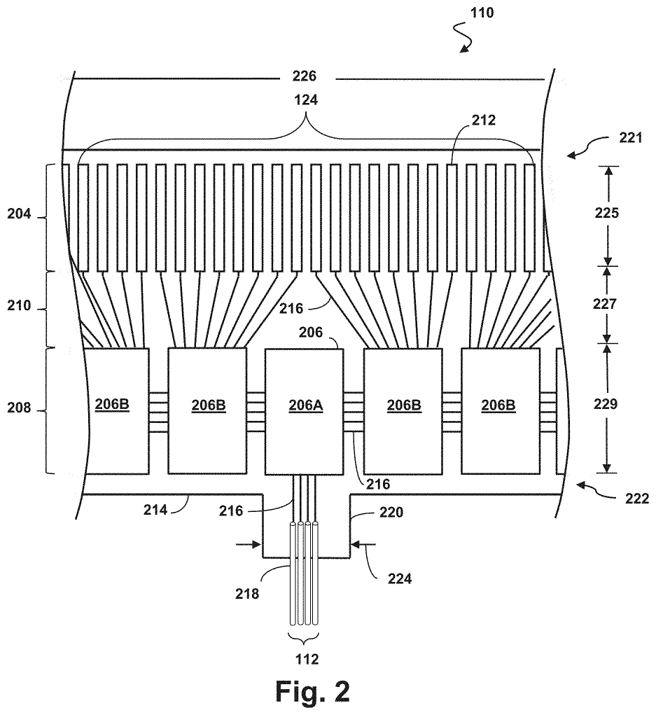

FIG. 2 is a diagrammatic top view of an ultrasound imaging assembly 110 in a flat configuration, according to aspects of the present disclosure. The flexible assembly 110 includes a transducer array 124 formed in a transducer region 204 and transducer control logic dies 206 (including dies 206A and 206B) formed in a control region 208, with a transition region 210 disposed therebetween. The transducer array 124 includes an array of ultrasound transducer elements 212. The transducer control logic dies 206 are mounted on a flexible substrate 214 into which the transducer elements 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

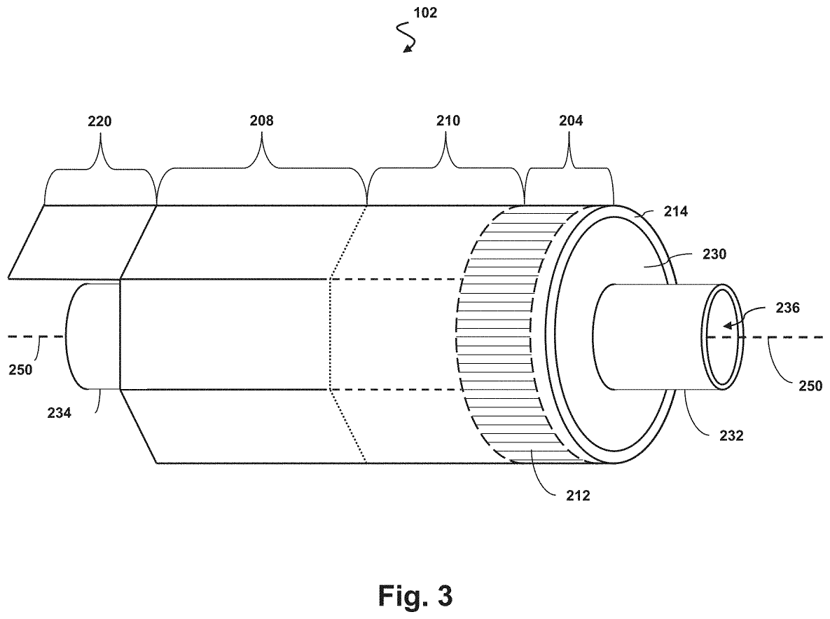

The flexible substrate 214, on which the transducer control logic dies 206 and the transducer elements 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 μm, e.g., 6 μm.

The set of transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 112, between a processing system, e.g., processing system 106, and the flexible assembly 110. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a plurality of transducer elements 512 positioned on a transducer element 212 to emit an ultrasonic signal and selects a transducer element 212 to receive an echo. In the depicted embodiment, the master controller 206A does not directly control any transducer elements 212. In other embodiments, the master controller 206A drives the same number of transducer elements 212 as the slave controllers 206B or drives a reduced set of transducer elements 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducer elements 212, in an embodiment, the flexible substrate 214 includes conductive traces 216 formed in the film layer that carry signals between the control logic dies 206 and the transducer elements 212. In particular, the conductive traces 216 providing communication between the control logic dies 206 and the transducer elements 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace or pad.

The flexible substrate 214 can include a conductor interface 220 in some embodiments. The conductor interface 220 can be in a location of the flexible substrate 214 where the conductors 218 of the cable 112 are coupled to the flexible substrate 214. For example, the bare conductors of the cable 112 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 is a diagrammatic perspective view of the ultrasound imaging assembly 102 shown in FIG. 2 in a rolled configuration around a support member, according to aspects of the present disclosure. FIG. 3 illustrates a perspective view of the scanner assembly 110 in a rolled configuration. In some instances, the flexible substrate 214 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

Depending on the application and embodiment of the presently disclosed invention, transducer elements 212 may be piezoelectric transducers, single crystal transducer, or PZT (lead zirconate titanate) transducers. In other embodiments, the transducer elements of transducer array 124 may be flexural transducers, piezoelectric micromachined ultrasonic transducers (PMUTs), capacitive micromachined ultrasonic transducers (CMUTs), or any other suitable type of transducer element. In such embodiments, transducer elements 212 may comprise an elongate semiconductor material or other suitable material that allows micromachining or similar methods of disposing extremely small elements or circuitry on a substrate.

In some embodiments, the transducer elements 212 and the controllers 206 can be positioned in an annular configuration, such as a circular configuration or in a polygon configuration, around a longitudinal axis 250 of a support member 230. It is understood that the longitudinal axis 250 of the support member 230 may also be referred to as the longitudinal axis of the scanner assembly 110, the flexible elongate member 121, or the device 102. For example, a cross-sectional profile of the imaging assembly 110 at the transducer elements 212 and/or the controllers 206 can be a circle or a polygon. Any suitable annular polygon shape can be implemented, such as one based on the number of controllers or transducers, flexibility of the controllers or transducers, etc. Some examples may include a pentagon, hexagon, heptagon, octagon, nonagon, decagon, etc. In some examples, the transducer controllers 206 may be used for controlling the ultrasound transducers 512 of transducer elements 212 to obtain imaging data associated with the vessel 120.

The support member 230 can be referenced as a unibody in some instances. The support member 230 can be composed of a metallic material, such as stainless steel, or a non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. In some embodiments, support member 230 may be composed of 303 stainless steel. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process or a micro injection molding process.

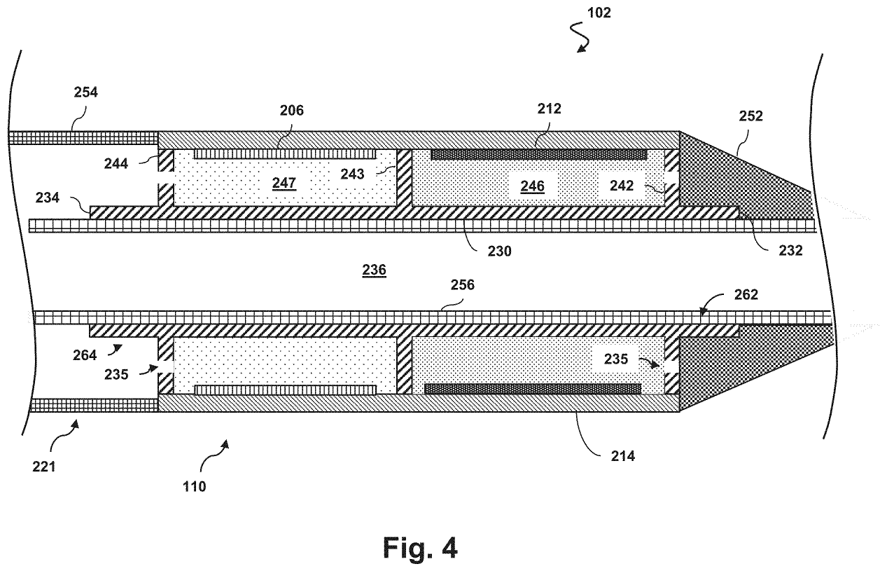

Referring now to FIG. 4, shown therein is a diagrammatic cross-sectional side view of a distal portion of the intraluminal imaging device 102, including the flexible substrate 214 and the support member 230, according to aspects of the present disclosure. The lumen 236 may be connected with the entry/exit port 116 and is sized and shaped to receive the guide wire 118 (FIG. 1). In some embodiments, the support member 230 may be integrally formed as a unitary structure, while in other embodiments the support member 230 may be formed of different components, such as a ferrule and stands 242, 243, and 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 243, and 244 that extend vertically are provided at the distal, central, and proximal portions respectively, of the support member 230. The stands 242, 243, and 244 elevate and support the distal, central, and proximal portions of the flexible substrate 214. In that regard, portions of the flexible substrate 214, such as the transducer portion 204 (or transducer region 204), can be spaced from a central body portion of the support member 230 extending between the stands 242, 243, and 244. The stands 242, 243, 244 can have the same outer diameter or different outer diameters. For example, the distal stand 242 can have a larger or smaller outer diameter than the central stand 243 and/or proximal stand 244 and can also have special features for rotational alignment as well as control chip placement and connection.

To improve acoustic performance, the cavity between the transducer array 212 and the surface of the support member 230 may be filled with an acoustic backing material 246. The liquid backing material 246 can be introduced between the flexible substrate 214 and the support member 230 via passageway 235 in the stand 242, or through additional recesses as will be discussed in more detail hereafter. The backing material 246 may serve to attenuate ultrasound energy emitted by the transducer array 212 that propagates in the undesired, inward direction.

The cavity between the circuit controller chips 206 and the surface of the support member 230 may be filled with an underfill material 247. The underfill material 247 may be an adhesive material (e.g. an epoxy) which provides structural support for the circuit controller chips 206 and/or the flexible substrate 214. The underfill 247 may additionally be any suitable material.

In some embodiments, the central body portion of the support member can include recesses allowing fluid communication between the lumen of the unibody and the cavities between the flexible substrate 214 and the support member 230. Acoustic backing material 246 and/or underfill material 247 can be introduced via the cavities (during an assembly process, prior to the inner member 256 extending through the lumen of the unibody. In some embodiments, suction can be applied via the passageways 235 of one of the stands 242, 244, or to any other suitable recess while the liquid backing material 246 is fed between the flexible substrate 214 and the support member 230 via the passageways 235 of the other of the stands 242, 244, or any other suitable recess. The backing material can be cured to allow it to solidify and set. In various embodiments, the support member 230 includes more than three stands 242, 243, and 244, only one or two of the stands 242, 243, 244, or none of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions of the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can comprise a flexible elongate member. The proximal inner member 256 can be received within a proximal flange 234. The proximal outer member 254 abuts and is in contact with the proximal end of flexible substrate 214. A distal tip member 252 is coupled to the distal portion 262 of the support member 230. For example, the distal member 252 is positioned around the distal flange 232. The tip member 252 can abut and be in contact with the distal end of flexible substrate 214 and the stand 242. In other embodiments, the proximal end of the tip member 252 may be received within the distal end of the flexible substrate 214 in its rolled configuration. In some embodiments there may be a gap between the flexible substrate 214 and the tip member 252. The distal member 252 can be the distal-most component of the intraluminal imaging device 102. The distal tip member 252 may be a flexible, polymeric component that defines the distal-most end of the imaging device 102. The distal tip member 252 may additionally define a lumen in communication with the lumen 236 defined by support member 230. The guide wire 118 may extend through lumen 236 as well as the lumen defined by the tip member 252.

One or more adhesives can be disposed between various components at the distal portion of the intraluminal imaging device 102. For example, one or more of the flexible substrate 214, the support member 230, the distal member 252, the proximal inner member 256, the transducer array 212, and/or the proximal outer member 254 can be coupled to one another via an adhesive. Stated differently, the adhesive can be in contact with e.g. the transducer array 212, the flexible substrate 214, the support member 230, the distal member 252, the proximal inner member 256, and/or the proximal outer member 254, among other components.

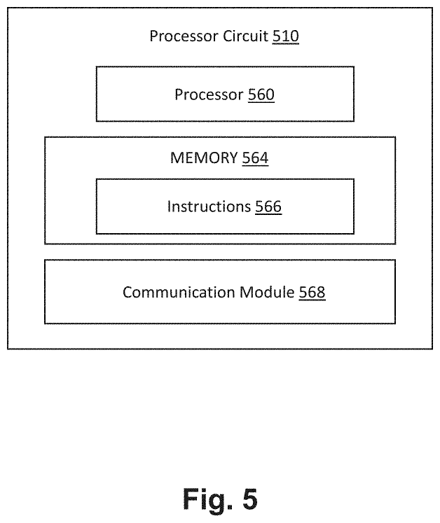

FIG. 5 is a schematic diagram of a processor circuit 510, according to aspects of the present disclosure. The processor circuit 510 may be implemented in the control system 130 of FIG. 1, the intraluminal imaging system 101, and/or the x-ray imaging system 151, or any other suitable location. In an example, the processor circuit 510 may be in communication with intraluminal imaging device 102, the x-ray imaging device 152, the display 132 within the system 100. The processor circuit 510 may include the processor 134 and/or the communication interface 140 (FIG. 1). One or more processor circuits 510 are configured to execute the operations described herein. As shown, the processor circuit 510 may include a processor 560, a memory 564, and a communication module 568. These elements may be in direct or indirect communication with each other, for example via one or more buses.

The processor 560 may include a CPU, a GPU, a DSP, an application-specific integrated circuit (ASIC), a controller, an FPGA, another hardware device, a firmware device, or any combination thereof configured to perform the operations described herein. The processor 560 may also be implemented as a combination of computing devices, e.g., a combination of a DSP and a microprocessor, a plurality of microprocessors, one or more microprocessors in conjunction with a DSP core, or any other such configuration.

The memory 564 may include a cache memory (e.g., a cache memory of the processor 560), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 564 includes a non-transitory computer-readable medium. The memory 564 may store instructions 566. The instructions 566 may include instructions that, when executed by the processor 560, cause the processor 560 to perform the operations described herein with reference to the probe 110 and/or the host 130 (FIG. 1). Instructions 566 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 568 can include any electronic circuitry and/or logic circuitry to facilitate direct or indirect communication of data between the processor circuit 510, the probe 110, and/or the display 132 and/or display 132. In that regard, the communication module 568 can be an input/output (I/O) device. In some instances, the communication module 568 facilitates direct or indirect communication between various elements of the processor circuit 510 and/or the probe 110 (FIG. 1) and/or the host 130 (FIG. 1).

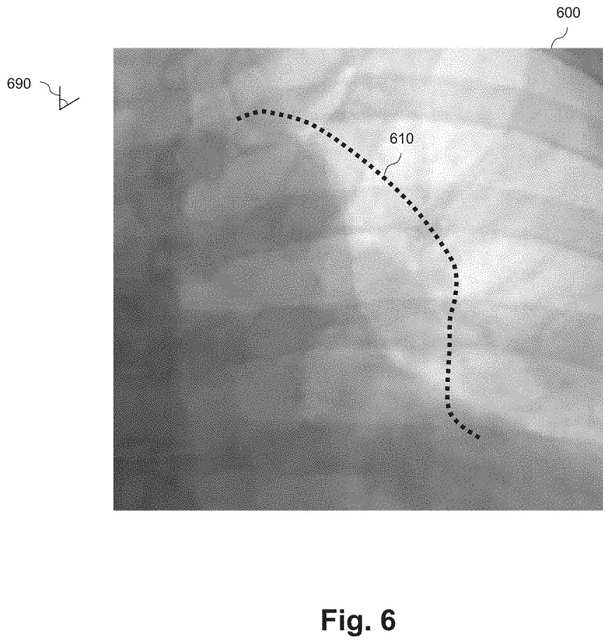

FIG. 6 is a diagrammatic view of an x-ray image 600, according to aspects of the present disclosure. As previously mentioned, one objective of the present disclosure may be to perform a coregistration procedure between intraluminal data, such as IVUS data or physiology data, with an extraluminal image without introducing contrast to a patient.

At a high level, a coregistration procedure involves performing an intravascular procedure and an extraluminal imaging procedure simultaneously. For example, a patient anatomy may be positioned within an imaging region of an extraluminal imaging device. The extraluminal imaging device may acquire extraluminal images of the patient. While extraluminal images are acquired, a physician may position an intraluminal device, such as an IVUS catheter, within a vessel of a patient within the view of the extraluminal imaging device. As the physician moves the IVUS catheter through the vessel, a radiopaque portion of the IVUS device may be observed within the x-ray images. In that regard, in each received x-ray image, the position of the IVUS device may be in a different location as the device moves through the vessel. As the IVUS device moves, it may acquire IVUS images. Because the IVUS images and x-ray images are acquired simultaneously, the system may correspond an IVUS image with the position of the IVUS device at that time as observed in an x-ray image. The many positions of the IVUS device during this procedure may be stored as a series of coordinates and may be used to determine a pathway of the device as it moved through the vessel. Each location along the generated pathway may then correspond to an IVUS image. In many coregistration procedures, this pathway is then overlaid over an additional x-ray image with contrast. To obtain this x-ray image, a physician may administer a contrast agent to the vasculature of the patient. This contrast agent may cause the vessels within the x-ray images to appear. Without a contrast agent introduced, a typical x-ray image may not show any blood vessels. The pathway, which is generated based on the locations of the IVUS device, may be overlaid on an x-ray image with contrast. Or, in some cases, a centerline based on the positions of a vessel within multiple x-ray images with contrast, to ensure that the IVUS pathway matches the correct vessel and that the coregistration of IVUS data to the x-ray image with contrast (e.g., an angiogram) is accurate.

Although an intravascular ultrasound device has been described in the example above, the same principles may apply to any suitable intraluminal procedure. For example, other intraluminal data, such as physiology data including blood pressure data (e.g., FFR data, iFR data, or other pressure data) or blood flow data, may also be acquired and coregistered to an extraluminal image in the same way.

In some cases, some patients may be more sensitive to the contrast agents used to make blood vessels appear in x-ray images. In particular, patients with Chronic Kidney Disease (CKD) may be more susceptible to developing complications from the use of contrast agents. For example, patients may be exposed to a risk of Contrast Induced Nephropathy (CIN) if contrast agent is introduced to their vasculature. The present invention advantageously provides a way of performing a coregistration step without the use of a contrast agent for representing the roadmap image or the use of a much lower dose of contrast agent. This advantageously leads to patients' risk of complications related to contrast agent exposure dramatically decreasing. This may allow patients to be released from coregistration procedures sooner, may reduce the procedure time of procedures themselves, and may lead to same-day discharge for more patients, even after the most complex interventions.

The extraluminal image 600 shown in FIG. 6 may be a view of a patient's anatomy. The image 600 may be an x-ray image obtained without a contrast agent introduced to the vasculature. This may be seen because no blood vessels are observed within the image 600. As an example, a pathway 610 is shown overlaid over the image 600. This pathway 610 may correspond to a vessel within the anatomy. Specifically, the pathway 610 may correspond generally to the motion of an intraluminal device during an intraluminal procedure. However, the pathway 610 may not be visible within the image 600 as originally acquired because the the pathway 610 is a calculated line overlaid on the extraluminal image. In some embodiments, the system will automatically distinguish between an angiogram that has contrast (standard angiogram) to an angiogram without contrast (zero contrast angiogram).

In some embodiments, the image 600 may alternatively be a low contrast image or ultra-low contrast image. An ultra-low contrast image 600 may be an x-ray image obtained with less than 20 cc of contrast introduced to the vasculature. A low contrast image may be an image obtained with a greater quantity of contrast agent used.

In some embodiments, the processor circuit 510 may receive and store in a memory in communication with the circuit 510 an angle 690 and a zoom setting of the extraluminal imaging device. The angle 690 may correspond to an angle of a c-arm relative to the patient anatomy when the image 600 was acquired. The zoom setting may correspond to the amount of zoom the extraluminal imaging system used, if any, while acquiring the image 600. This information may be provided to a user or used by the processor circuit 510 in subsequent procedures. Once a zero-contrast angiogram is identified, the co-registration workflow, user display, and calculations will be optimized for this scenario according to elements of the invention described herein. For example, a processor circuit (e.g., the processor circuit 510 of FIG. 5) may be configured to display prompts or labels for a user on the display (e.g., the display 132) to guide a user in acquiring necessary data and performing necessary procedures to perform zero-contrast coregistration.

The extraluminal image 600 may be any suitable type of extraluminal image. For example, the image 600 may be a cine image obtained without contrast or a fluoroscopy image obtained without contrast. In some embodiments, a cine image may correspond to an x-ray image obtained with a relatively higher dose of radiation or a relatively higher frame rate, and thus may be an image with relatively higher resolution. In some embodiments, a fluoroscopy image is an x-ray image obtained with a relatively lower dose of radiation or a relatively lower frame rate and may be an image with relatively lower resolution.

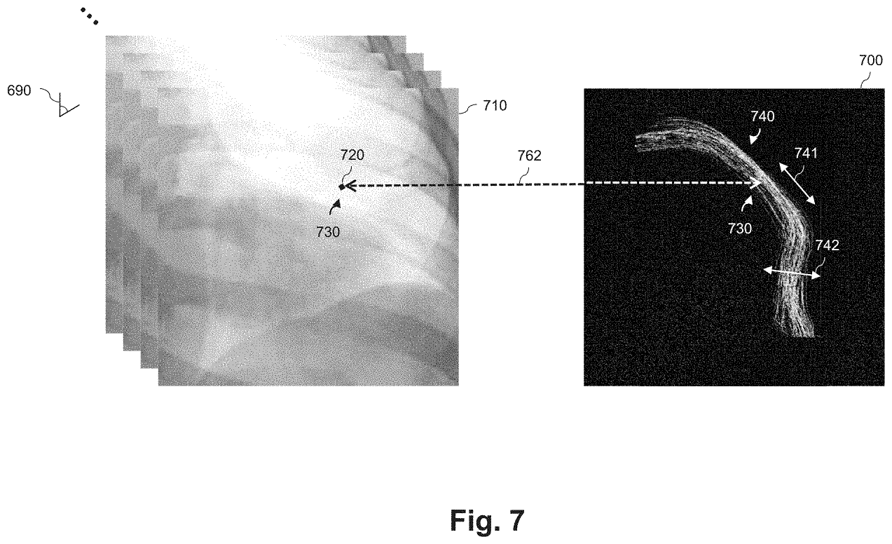

It is noted that the pathway 610 shown in FIG. 6 may be a motion-corrected path. For example, as will be explained in more detail with reference to FIGS. 7-11 below, various techniques may be used to account for movement of the patient anatomy during imaging. For example, in an example in which a coronary vessel is imaged by the IVUS imaging device, the patient anatomy imaged as shown in the image 600 may be constantly moving as the heart beats. As a result, the radiopaque portions of the IVUS device may be constantly moving in a cyclical pattern with the beating heart. As a result, the observed movement of the IVUS device may not initially appear as the pathway 610 shown in FIG. 6, but may be a set of locations showing movement in one or more directions.

FIG. 7 illustrates a relationship between multiple extraluminal images 710 and a set of locations 740, according to aspects of the present disclosure. At one step of the present disclosure, such as during an intraluminal pullback procedure, the processor circuit 510 may be configured to receive multiple extraluminal images 710. The multiple extraluminal images 710 may be obtained by the extraluminal imaging system 151. The extraluminal images 710 may display one or a plurality of radiopaque portions of an intraluminal sensing device 720 (e.g., an intravascular imaging catheter, an intravascular pressure-sensing guidewire, etc.). As the device is moved through the patient vasculature, each consecutive extraluminal image 710 may depict the radiopaque portion of the intraluminal device 720 in a different location within the image. These locations of the radiopaque portion of the intraluminal imaging device 720, may be stored in a memory in communication with the processor circuit 510. These locations may be stored as coordinates of pixels within an image. For example, as shown in the image 710 of FIG. 7, the device 720 may be observed within the image 710 at a location 730. A coordinate corresponding to the location 730 may be stored in the memory in association with the image 710. In some embodiments, all of the images 710 may be obtained with the extraluminal imaging system 151 at the same angle 690 and zoom setting as was used to obtain the image 600 of FIG. 6. In some embodiments, the image 600 can be chosen as one of the images 710.

All of the coordinates of the positions of the device 720 in all of the received images 710 may create a set of locations 740. The image 700 shown in FIG. 7 may depict the set of locations 740. The set of locations 740 may identify a plurality (e.g., some, all, or substantially all) of the locations of one or more radiopaque portions of the device 720 as it traveled through a vessel of the patient anatomy. As shown in FIG. 7, the set of locations 740 may include a direction 741 parallel to the pathway 740 and a direction 742 perpendicular to the direction of the pathway 740. In some embodiments, the direction 741 can correspond to the direction of movement of the device through the vessel as it collects intraluminal data (e.g., intravascular images, intravascular pressure, etc.), such as during a pullback. As shown in FIG. 7, and specifically in the image 700, the location of the device 720 may include various positions both in a parallel direction 741 and a perpendicular direction 742. In some embodiments, during an imaging procedure, the imaged vessel may move as the anatomy of the patient moves. For example, if a vessel within a heart of a patient is imaged, throughout an imaging procedure, the heart may continuously pump blood to the rest of the anatomy of the patient. In this example, various vessels of the heart, including a vessel of the heart in which the intraluminal device 720 is positioned, will move as the various muscles of the heart move. These muscle movements may account for variations in the position of the intraluminal device 720 in either a perpendicular direction 742 or a parallel direction 741.

As shown by the arrow 762, positions observed within the images 710 may be identified or associated with the set of locations 740 in the image 700. In some embodiments, the image 700 is a composite image showing the locations of the radiopaque portions from a plurality (e.g., some, all, or substantially all) of the images 710. In this way, data collected at various locations of the device 720 by the device 720, such as IVUS images or physiology data, may be associated with corresponding locations in the set of locations 740. As an example, the location 730 shown in the image 710 may also be identified in the image 700. Aspects of determining and displaying the pathway that the device travels through the vessel are described in U.S. application Ser. No. 15/630,482, filed Jun. 22, 2017, and titled, “Estimating the endoluminal path of an endoluminal device along a Lumen,” which is hereby incorporated by reference in its entirety.

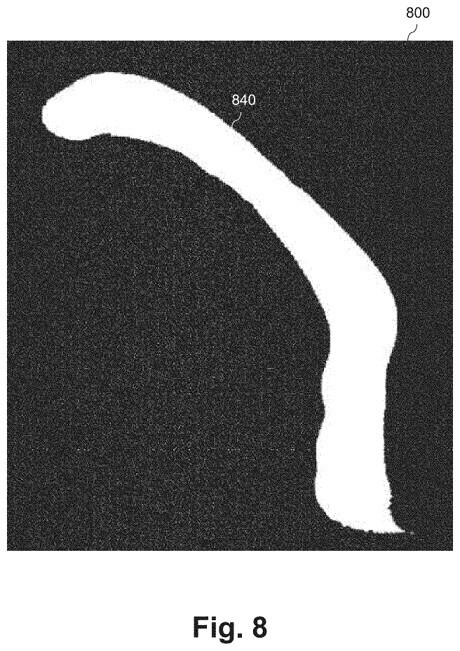

FIG. 8 is a diagrammatic view of a shape 840 based on the set of locations 740, according to aspects of the present disclosure. In some embodiments, the shape 840 is formed to include only the set of locations 740. The shape 840 may be a closed shape in some embodiments. This shape 840 may be displayed within an image 800. This may be done by any suitable image processing techniques. For example, the processor circuit 510 may identify the regions of the image 700 corresponding to the set of locations 740. For example, the processor circuit 510 may be configured to identify an outer edge of all of the pixel coordinates which together define the set of locations 740. This outer edge may define the shape 840. The processor circuit 510 may employ any suitable image processing techniques. For example, the system 100 may use image processing techniques such as edge detection, image editing or restoration, linear filtering or other filtering methods, image padding, or any other suitable image processing techniques. For example, the system 100 can use a pixel-by-pixel analysis to identify longitudinally adjacent dark pixels within the image 700. In some embodiments, the system 100 may use deep learning techniques to identify the locations of the outer edges of the shape 840.

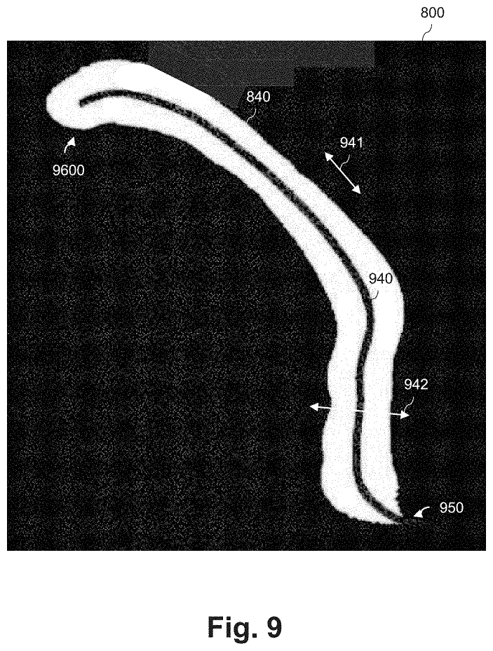

FIG. 9 is a diagrammatic view of a calculated footprint line 940 of the shape 840 based on the movement of an intraluminal device, according to aspects of the present disclosure. FIG. 9 includes the image 800 with the shape 840 and an identified calculated footprint line 940. The calculated footprint line 940 may also be referred to as a calculated footprint line, a corrected footprint line, a pathway, a corrected pathway, a centerline, a corrected centerline, a motion-corrected pathway, a motion-corrected footprint line, a motion-corrected centerline, or any other term. In some embodiments, the processor circuit 510 may identify various directions associated with the shape 840. For example, a direction 941 may correspond to a parallel direction of the shape 840 along the length of the shape 840. In addition, a direction 942 may correspond to a perpendicular direction of the shape 840.

In some embodiments, the processor circuit 510 may be configured to calculate a width of the shape 840 at all locations of the shape 840. For example, beginning at a distal position 950 of the shape 840, The processor circuit may determine a width in a perpendicular direction 942 at each location along the shape 840 to proximal location 960. The calculated footprint line 940 may then be calculated based on these width measurements along the length of the shape 840. For example, at each location along the shape 840, the processor circuit may determine a width and position the calculated footprint line 940 at a distance of half of this width from either of the outer edges of the shape 840. This calculated footprint line 940 may represent the pathway that the intravascular device traveled through the blood vessel corrected for motion. In that regard, the calculated footprint line 940 may be an illustration of movement the intravascular device through the patient anatomy if the patient anatomy had remained stationary during the imaging procedure. Because the intravascular device is inside the blood vessel as it moves through the blood vessel, the calculated footprint line 940 may also be a representation of the shape and position of the imaged vessel. Thus, the shape and position of the imaged vessel without using contrast in x-ray frames may be calculated. In the case of a no-contrast angiogram, the algorithm of the present disclosure may map the estimated luminal path (e.g., the calculated footprint line 940) to a non-visible vessel contour. This may require the calculated footprint line to conform to a non-visible centerline of the blood vessel imaged by the IVUS device. As a result, the presumed mapping of the calculated footprint line to a vessel centerline may introduce inaccuracies which are corrected according to principles of the disclosure described herein.

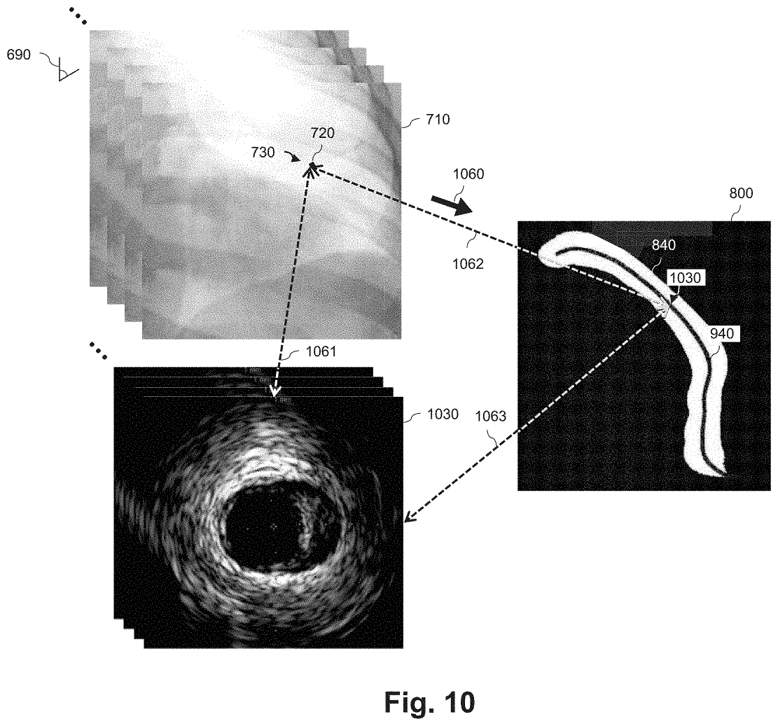

FIG. 10 illustrates a relationship between IVUS data 1030, extraluminal images 710, and the calculated footprint line 940. As described with reference to FIG. 7, the set of locations 740 shown in FIG. 7 corresponds to the locations of the intravascular imaging device 720 as it moved through a vessel during an imaging procedure. The calculated footprint line 940 is a simplified view of the set of locations 740 that is motion corrected. Each position of the device 720 in the images 710 correspond to a position both among the set of locations 740 and the calculated footprint line 940. In that regard the location 730 of the set of locations 740, described with reference to FIG. 7, may correspond to a similar position 1030 along the calculated footprint line 940. This relationship may be shown by the arrow 1062. Similarly, each position of the device 720 in the images 710 may be associated with one of the plurality of IVUS images 1030. In some embodiments, data associated with the locations of the device 720 in the images 710 may be other intraluminal data. For example, as has been previously described, intraluminal data 1030 may include IVUS images as shown in FIG. 10, physiology data such as pressure data or flow data, or any other suitable intraluminal data. As shown by the arrow 1061, each intraluminal datum 1030, such as the IVUS image 1030 shown in FIG. 10, may be associated with at least one location within at least one extraluminal image 710. Based on the relationship between the images 710 and the calculated footprint line 940 shown in FIG. 10 by the arrow 1062, the intraluminal data 1030 associated with the locations of the device 720 may be similarly associated with locations along the calculated footprint line 940. For example, the first IVUS image 1030 shown in FIG. 10 may be associated with the location 730 within the first x-ray image 710 as shown by the arrow 1061. That same first IVUS image 1030 shown in FIG. 10 may also be associated with the location 1030 of the calculated footprint line 940 as shown by the arrow 1063. A similar relationship may exist for all IVUS images 1030, or other intraluminal data, all extraluminal images 710, and all locations along the calculated footprint line 940.

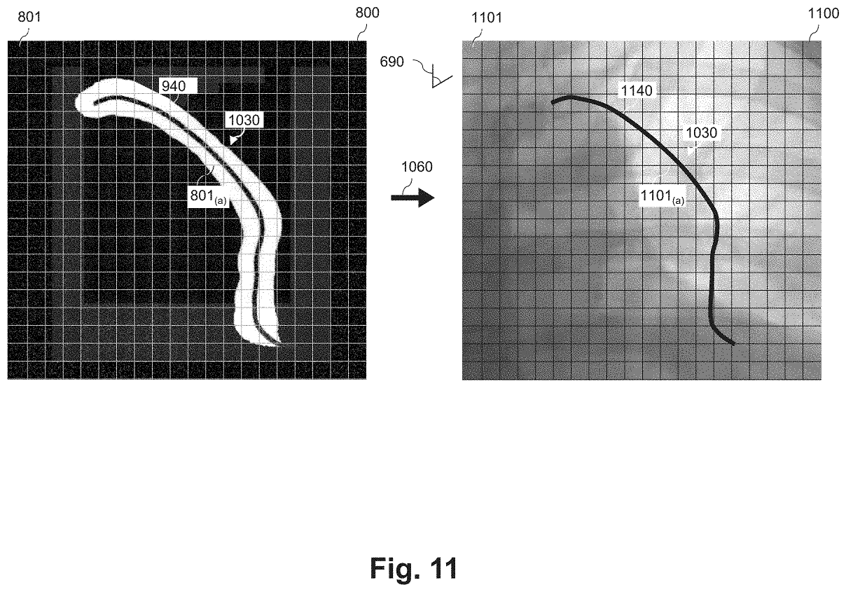

FIG. 11 illustrates a relationship between the calculated footprint line 940 and coregistered intraluminal data with a calculated centerline1140 overlaid over an extraluminal image 1100, according to aspects of the present disclosure.

In some embodiments the calculated footprint line 940 may be defined by multiple pixel coordinates within an image 800. For example, the image 800 may include or be made of multiple pixels. As an illustration of these pixels, the image 800 may be divided into multiple boxes 801. Each box 801 may correspond to a pixel of the image. The number of boxes 801 representing pixels of the image 800 as shown in FIG. 11 may be any suitable number. The arrangement and number of boxes 801 shown in the image 800 of FIG. 11 is only illustrative and for pedagogical purposes. For example, the image 800 may include more or less pixels than those illustrated by the boxes 801 shown in FIG. 11.

In some embodiments, the processor circuit 510 may be configured to receive an additional extraluminal image 1100. The extraluminal image 1100 may be any suitable extraluminal image. For example, the extraluminal image 1100 may be an x-ray image. In one example, the extraluminal image 1100 may be an x-ray image obtained without contrast, such as a fluoroscopy image or a cine image. In some embodiments, the x-ray image 1100 may be the same size as the image 800. For example, the image 1100 may contain the same number of pixels in the same arrangement and of the same resolution as the pixels of the image 800. In addition, the angle and zoom of the extraluminal imaging system used to acquire the image 1100 may match the angle and zoom used to acquire the images 710. This same angle may be denoted by the angle 690 shown adjacent to the image 1100. In some embodiments, the image 800 containing the calculated footprint line 940 may correspond to the same angle 690 and zoom settings of the images 710 from which it was derived as well as the image 1100. As a result, there may be a one-to-one correspondence of pixels 801 within the image 800 to pixels 1101 in the image 1100. In that regard, a location within a patient anatomy represented by a single pixel 801 within the image 800 may also be represented by a corresponding pixel 1101 in FIG. 1100.

As an example of the correspondence between pixels 801 of the image 800 and pixels 1101 of the image 1100, a location 1030 is shown within each image 800 and 1100. This location 1030 may be a location along the pathway 940 of image 800 and along the calculated centerline 1140 of the image 1100. In the image 800, this location 1030 may be identified by, or correspond to, the pixel 801₍ₐ₎. The same location 1030 may be identified by, or correspond to, the pixel 1101₍ₐ₎. This relationship between the image 800 and the calculated footprint line 940, and the image 1100 and the calculated centerline 1140 may be signified by the arrow 1060 shown in FIG. 11.

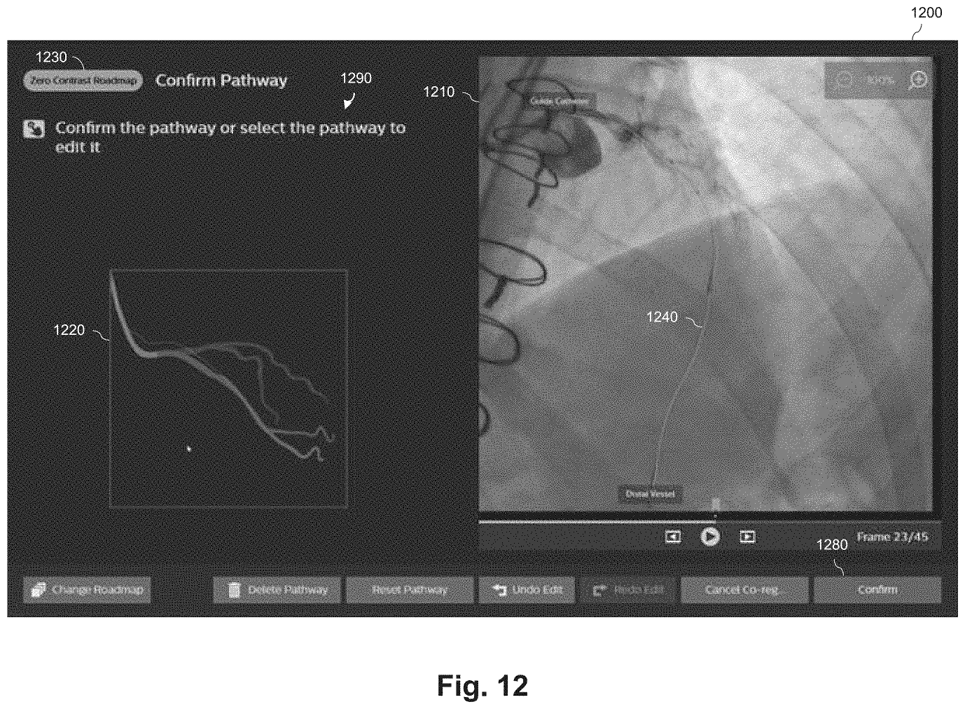

FIG. 12 is a diagrammatic view of a graphical user interface 1200, according to aspects of the present disclosure. In some embodiments, the graphical user interface 1200 may be displayed to a user of the system after the steps described in FIGS. 7-11 are complete. For example, a system may track an intraluminal device in multiple fluoroscopy images to create a pathway (e.g., the pathway 740 of FIG. 7). The system may then convert the pathway 740 to an calculated footprint line 940 as described with reference to FIG. 8 and FIG. 9. The system may then display the calculated footprint line 940 overlaid over an additional extraluminal image obtained at the same angle and zoom of the extraluminal images obtained during a pullback procedure, as described with reference to FIG. 11.

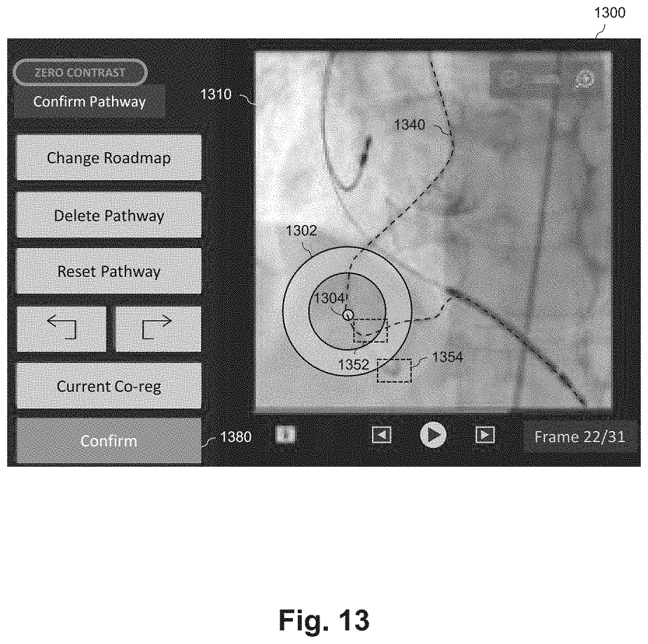

The extraluminal image 1210 may be an additional extraluminal image similar to the image 1100 described with reference to FIG. 11. As shown in FIG. 12, the image 1210 may include a depiction of a calculated footprint line1240 overlaid over the image 1210. This calculated footprint line 1240 may be generated by the processor circuit 510 according to methods described with reference to FIGS. 7-11. After the calculated footprint line 1240 is shown overlaid over the image 1210, the system 100 may prompt a user to either edit the calculated footprint line 1240 so as to adapt the calculated footprint line to a user-defined vessel centerline, as will be described in more detail hereafter (e.g., with reference to FIG. 13), or to confirm the pathway 1240. In this regard, the calculated footprint line 1240 may serve as a roadmap for the final co-registration calculation.

As shown in FIG. 12, the processor circuit may be configured to provide a button 1280, or other input element, by which a user may provide a user input indicating that the calculated footprint line1240 conforms to the vessel centerline. In this way, after the IVUS or physiology pullback and once the angiogram is obtained (either before or after the pullback), the system will automatically switch the user display to “semi-automated” mode so that the user is able to edit the generated calculated footprint lineto create a roadmap for the co-registration calculation and display. Aspects of confirming, editing, or estimating the calculated footprint line may include any features or characteristics as those described in E.P. Patent No. 3474750B1, “Estimating the Endoluminal Path of an Endoluminal Device Along a Lumen,” filed Jun. 22, 2016, the entirety of which is hereby incorporated by reference herein.

According to some aspects of the present disclosure, a user of the system may confirm the shape of the calculated footprint line 1240 based on a number of references. For example, a user of the system may verify that the calculated footprint line 1240 accurately resembles the expected shape of the vessel by comparison to a contrast-filled angiogram of the same patient anatomy. For example, in some embodiments, during a previous procedure, a contrast agent may have been introduced into the patient vasculature. For example, a contrast agent may have been introduced into the patient vasculature in combination with the positioning of an initial guidewire, such as a workhorse guidewire, or a guidewire of an IVUS imaging device or other intraluminal device. In some embodiments, the contrast agent introduced may have been a low dose or ultra-low dose. In some embodiments, a low dose or ultra-low dose may correspond to a dose of 5 mL or 5 cc of contrast agent of any of the materials listed previously. In some embodiments, an extraluminal image acquired by the extraluminal imaging device 151 while contrast is present within the patient vasculature may be stored by the processor circuit 510 in a memory in communication with the processor circuit 510. When the processor circuit 510 prompts the user to confirm the shape and position of the calculated footprint line 1240, as shown in FIG. 12, the processor circuit 510 may be additionally configured to retrieve and display this extraluminal image with contrast (e.g., an angiogram, such a selected frame from a cine series of frames with or without contrast injection) and simultaneously display this image to the user along with the image 1210 and calculated footprint line 1240. The user of the system 100 may then compare the shape and position of the calculated footprint line 1240 with the angiogram from the previous procedure stored in the memory and confirm whether the calculated footprint line 1240 matches the vessel centerline of the target vessel in the angiogram.

In another embodiment, the user of the system 100 may confirm the shape of the calculated footprint line 1240 by comparing it to the observed path of the intravascular device during the intraluminal procedure used for coregistration described with reference to FIG. 7. For example, the user may confirm that the calculated footprint line 1240 resembles the path as observed by the user during this step, as described in FIG. 7. In some embodiments, the processor circuit 510 may retrieve any or all of the images 710 (FIG. 7) and display them to a user within the graphical user interface 1200 for comparison. In one example, the processor circuit 510 may be configured to display the images 710 in rapid succession and in chronological order to replay the movement of the device within the images 710. The user may confirm, based on the comparison of the shape and position of the calculated footprint line 1240 with the movement of the intraluminal device in the images 710, that the shape and position of the calculated footprint line 1240 is accurate.

In another embodiment, the user of the system 100 may confirm the shape and position of the calculated footprint line 1240 by referencing anatomical or other landmarks within the image 1210 that were observed previously. For example, the user may observe anatomical landmarks including various bone structures, abnormalities in bone structures or other anatomies of the patient, or any other anatomical landmarks during an initial imaging stage (e.g., the intraluminal imaging phase described with reference to FIG. 7). In some embodiments, landmarks may also include man-made structures such as stents, other treatment devices, clips, or any other structures. In some embodiments, the user of the system may identify any of these structures during an initial imaging procedure as well as within the image 1210 and be able to judge, based on the location of the calculated footprint line 1240 to these landmarks, the accuracy of the shape and position of the calculated footprint line 1240. In some embodiments, the processor circuit may be configured to receive user inputs during an initial imaging stage of landmarks within the images 710. The locations of these images may be stored in a memory in communication with the processor circuit 510 and displayed in the same location, based on a stored pixel coordinate, in the image 1210 to assist a user in comparing and confirming the calculated footprint line 1240. In some embodiments, the processor circuit 510 may be configured to automatically identify various landmarks and display them to a user in either the image 710 and/or the image 1210.

In some embodiments, the user of the system 100 may confirm the shape and position of the calculated footprint line 1240 by comparing the calculated footprint line 1240 to a no-contrast extraluminal image of the patient anatomy obtained while multiple guidewires are positioned within one or more vessels of the patient. In that regard, the radiopaque portions of the multiple guidewires highlight the vessel profile. In some embodiments, this image of the patient anatomy obtained with multiple guidewires within the anatomy may be obtained during the same imaging procedure as the procedure obtaining the multiple IVUS images and/or extraluminal images described herein. In some embodiments, the image may have been obtained during a previous procedure and may be retrieved from a memory.

In some embodiments, the processor circuit 510 may be configured to display various prompts to the user. For example, a prompt 1290 may direct a user to confirm the shape and position of the calculated footprint line 1240 by, for example, selecting a button 1280. The prompt 1290 may additionally convey that a user may edit the calculated footprint line 1240 by clicking on the calculated footprint line 1240 within the image 1210, as will be described in more detail with reference to FIG. 13. In some embodiments, a prompt 1220, or symbol or image 1220, may quickly convey to the user that the user may adjust the position or shape of the calculated footprint line 1240.

An indicator 1230 may be provided in the screen display 1200 identifying for the user that the x-ray image is obtained without contrast, and thus the user is confirming a zero-contrast roadmap. The Co-Registration results screen (e.g., the interface 1200, or other interfaces described in the figures described hereafter) are labelled as zero contrast as demonstrated by the indicator 1230. The labelling of the display 1200 as zero contrast and associated workflow will be clearly evident to any observer.

According to another aspect of the present disclosure, the calculated footprint line 1240 initially displayed to the user may be different from a calculated footprint line involving a contrast-based angiogram and may more closely match the intended roadmap,thus requiring less user editing because, for example, the algorithm for calculating and displaying the calculated footprint line does not require obtaining or identifying a contrast-filled vessel as also described in EP 3474750, incorporated by reference previously.

FIG. 13 is a diagrammatic view of a graphical user interface 1300, according to aspects of the present disclosure. In some embodiments, the graphical user interface 1300 may be displayed to a user after the user selects an input to edit a pathway, such as the calculated footprint line 1240 of FIG. 12.

In the example shown in FIG. 13, an extraluminal image 1310 is provided. The image 1310 may be similar to the image 1210 describe with reference to FIG. 12 and/or the image 1100 described with reference to FIG. 11. In some embodiments, the image 1310 may be an extraluminal image (e.g., an x-ray image) obtained without contrast introduced into a patient vasculature.

As shown in FIG. 13, the image 1310 may include a depiction of a calculated footprint line 1340. The calculated footprint line 1340 may be similar to the calculated footprint line 1240 previously described. In the example shown in FIG. 13, the user may wish to edit the shape and position of the calculated footprint line 1340 such that it matches a known shape and position of the vessel imaged. For example, the user may determine a desired or correct shape based on a previously acquired angiogram image (e.g., an x-ray image acquired with a contrast agent introduced to the vasculature), a view of the movement of the intraluminal device during a previous intraluminal procedure, nearby anatomical or man-made landmarks, or any other references. As shown in FIG. 13, the calculated footprint line 1340 may extend through a section 1352 of the image. The user of the system may be aware, based on any of the references previously described, that the calculated footprint line 1340 should actually match the shape shown in the region 1354 of the image. It is noted, that although a path may be visible within the region 1354, such as a path identified as a vessel with a contrast agent, this is displayed for pedagogical purposes only. In most implementations, in which no contrast agent is present, the desired or corrected location of any region of the calculated footprint line 1340 may not be visible to a user. However, the user may be aware of the corrected location based on the references described.

As shown in the graphical user interface 1300, the processor circuit 510 may be configured to provide, within the display, various user-selectable tools for editing the shape and/or location of the calculated footprint line 1340. For example, as shown in FIG. 13, an indicator 1302 may show an area of the calculated footprint line 1340 that the user modified. The indicator 1302 may or may not be displayed. In some embodiments, after the user has selected an input for the processor circuit 510 to enter a pathway shape editing mode, a user may select any location along the calculated footprint line 1340. In some embodiments, the user touch and drag (e.g., on a touchscreen display, using a mouse, etc.) the location on the calculated footprint line 1340 to a new position indicative of the correct shape of the calculated footprint line 1340. As an example, the user may select a location within region 1352 and move it to a location within the region 1354 representative of the correct shape of the calculated footprint line 1340. In response to the user input, the processor circuit 510 may be configured to modify the shape and/or position of the calculated footprint line 1340 such that it passes through the region 1354.

In some embodiments, this modifying of the shape and position of the calculated footprint line 1340 may include interpolation between anchors, such as an anchor 1304 and/or other anchors along the calculated footprint line 1340, defining the calculated footprint line 1340. In some embodiments, the interpolation may include a local interpolation. The anchor 1304 may or may not be displayed to a user. For example, only anchor points or regions of the calculated footprint line 1340 close in proximity to the moved anchor 1304 may be adjusted, while regions of the calculated footprint line 1340 far from the anchor 1304 may remain unchanged. In some embodiments, the indicator 1302 may define a region of proximity around the anchor 1304. Sections of the pathway 1304 within the region defined by the indicator 1302 may be modified while sections outside the anchor 1302 may be unchanged. In some embodiments, the user of the system 100 may adjust various settings or aspects of the interpolation algorithm, including, for example, the size and shape of the indicator 1302.

Some aspects of modifying the shape and position of the calculated footprint line 1340 may include features similar to those described in U.S. Provisional Application No. 63/187,964, titled, “PATHWAY MODIFICATION FOR COREGISTRATION OF EXTRALUMINAL IMAGE AND INTRALUMINAL DATA” and filed May 13, 2021 (International Publication No. WO 2022/238276), which is hereby incorporated by reference in their entirety.

In some embodiments, after the calculated footprint line 1340 has been modified to a user's satisfaction, the processor circuit 510 may receive an input indicating that the pathway is confirmed and the system may exit a pathway modification mode.

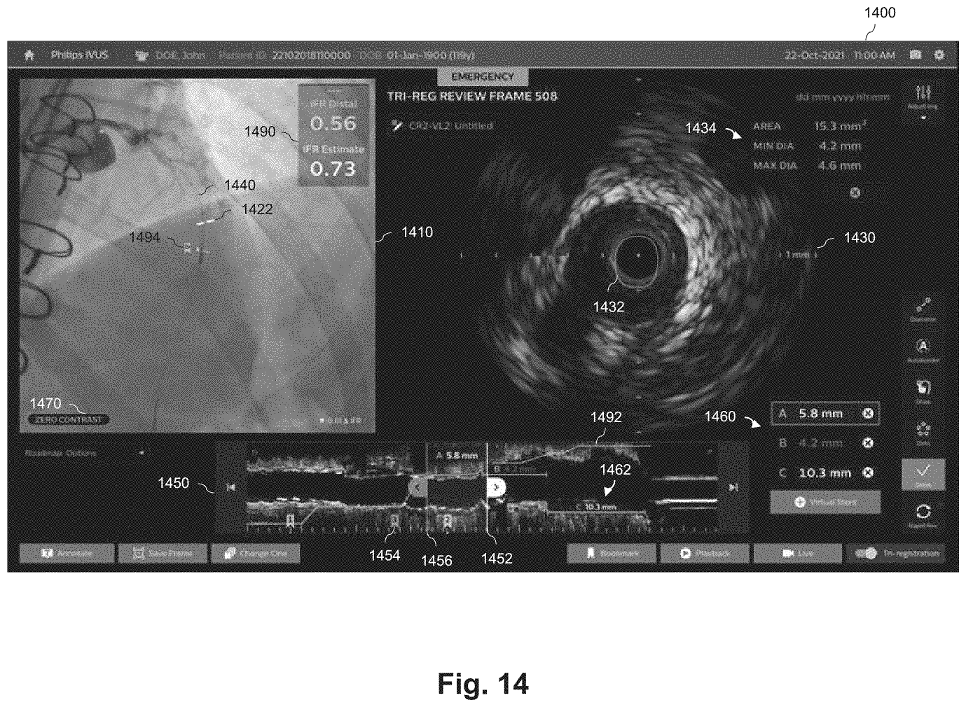

FIG. 14 is a diagrammatic view of a graphical user interface 1400. The graphical user interface 1400 may be displayed for a user after a pathway (e.g., the calculated footprint line 1340 of FIG. 13, the calculated footprint line 1240 of FIG. 12, and/or the centerline 1140 of FIG. 11) has been confirmed and/or modified.

After a pathway, including any of those previously mentioned and described, is confirmed and/or modified by the user, the processor circuit 510 may be configured to coregister any intraluminal data to the pathway. For example, as explained with reference to FIG. 7 and FIG. 10, intraluminal data, such as IVUS imaging data and/or physiology data, may be associated with locations along the confirmed pathway. When that pathway is overlaid over an extraluminal image, that intraluminal data may be displayed corresponding to locations within the extraluminal image illustrating where along a vessel, as shown by the pathway, that intraluminal data was acquired.

As an example, the graphical user interface 1400 provides an x-ray image 1410, an IVUS image 1430, physiology data 1490, and a longitudinal view 1450 of the imaged vessel. The x-ray image 1410 may include a depiction of a calculated footprint line 1440. The calculated footprint line 1440 may be similar to the centerline 1140 of FIG. 11, the pathway 1240 of FIG. 12, and/or the calculated footprint line 1340 of FIG. 13. In some embodiments, the calculated footprint line 1440 may be pathway corresponding to the movement of an intravascular imaging catheter that has been modified and/or confirmed by the user. The calculated footprint line 1440 may be overlaid over the image 1410 and may identify the location of the imaged blood vessel. Various indicators related to coregistered intraluminal data may be displayed along or next to this calculated footprint line 1440.

As an example, iFR data 1490 may be coregistered to the calculated footprint line 1440. For example, iFR data may be received by the processor circuit 510 during an iFR pullback while also receiving extraluminal images (e.g., the image 710 of FIG. 7). As iFR data are acquired and associated with locations within the extraluminal images, the iFR data may be identified at locations along the calculated footprint line 1440. As an example, an indicator 1422 may be provided along the calculated footprint line 1440. The indicator 1422 may correspond to the location along the calculated footprint line 1440 at which iFR data 1490, such as the iFR estimate metric, was acquired. Similarly, an indicator 1494 may be provided within the image 1410 along the calculated footprint line 1440. The indicator 1494 may identify the distal location that iFR data 1490 was acquired, such as the iFR distal value shown as part of data 1490.

Also shown within the graphical user interface 1400 is the IVUS image 1430. In that regard, a plurality of IVUS images (including image 1430) can be co-registered to the calculated footprint line 1440. The IVUS image 1430 may be an IVUS image obtained at the location identified by the indicator 1422. In some aspects, the indicator 1422 may also be referred to as a marking. The IVUS image 1430 may alternatively be an IVUS image obtained at the location identified by the indicator 1494. In some embodiments, the IVUS image 1430 may include a border 1432. This border may be identified automatically by the processor circuit 510 or may be identified by a user of the system. In some embodiments, the border 1432 may be a lumen border, a vessel border, a stent border, or any other border within the image.

Examples of border detection, image processing, image analysis, and/or pattern recognition include U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

Additionally depicted in the interface 1400 are metrics 1434. The metrics 1434 may relate to the IVUS image 1430 shown and specifically the border 1432. For example, the processor circuit 510 may automatically calculate various metrics 1434 related to the border 1432. For example, the processor circuit 510 may identify a cross-sectional area of the border 1432. The circuit may also identify a minimum diameter of the border, a maximum diameter of the border, or any other measurements or metrics related to the border 1432, or other aspects of the image 1430.

In some embodiments, the longitudinal view 140 may also be displayed. The longitudinal image 1450 may be referred to as in-line digital (ILD) display or intravascular longitudinal display (ILD) 1450. The IVUS images acquired during an intravascular ultrasound imaging procedure, such as during an IVUS pullback, may be used to create the ILD 1450. In that regard, an IVUS image is a tomographic or radial cross-sectional view of the blood vessel. The ILD 1450 provides a longitudinal cross-sectional view of the blood vessel. The ILD 1450 can be a stack of the IVUS images acquired at various positions along the vessel, such that the longitudinal view of the ILD 1450 is perpendicular to the radial cross-sectional view of the IVUS images. In such an embodiment, the ILD 1450 may show the length of the vessel, whereas an individual IVUS image is a single radial cross-sectional image at a given location along the length. In some embodiments, the ILD 1450 may illustrate a time at which IVUS images were obtained and the position of aspects of the ILD 1450 may correspond to time-stamps of the IVUS images. In another embodiment, the ILD 1450 may be a stack of the IVUS images acquired overtime during the imaging procedure and the length of the ILD 1450 may represent time or duration of the imaging procedure. The ILD 1450 may be generated and displayed in real time or near real time during the pullback procedure. As each additional IVUS image is acquired, it may be added to the ILD 1450. For example, at a point in time during the pullback procedure, the ILD 1450 shown in FIG. 9 may be partially complete. In some embodiments, the processor circuit may generate an illustration of a longitudinal view of the vessel being imaged based on the received IVUS images. For example, rather than displaying actual vessel image data, the illustration may be a stylized version of the vessel, with e.g., continuous lines showing the lumen border and vessel border. As shown in FIG. 11, the ILD 1450 may represent a stylized ILD shown the lumen border 1156 extending as continuous lines across the ILD 1450. The location of the lumen borders 1156 may be positioned symmetrically around a center axis and may be positioned according to the luminal diameter calculated in each corresponding IVUS image.

The ILD 1450 may include a depiction of iFR data 1492, various length measurements 1462, indicators 1452 and 1456 identifying the beginning and ending of a length measurement, and bookmark identifiers 1454. Aspects of providing physiology data (e.g., pressure ratio data such as a iFR data 1492) on the ILD 1450 are described in U.S. Provisional Application No. 63/288,553, filed Dec. 11, 2021, and titled “REGISTRATION OF INTRALUMINAL PHYSIOLOGICAL DATA TO LONGITUDINAL IMAGE OF BODY LUMEN USING EXTRALUMINAL IMAGING DATA”, which is incorporated by reference herein in its entirety.

In some embodiments, the iFR data 1492 may be the same iFR data used to populate the metrics 1490 described. As shown in the ILD 1450 and because the ILD 1450 is generated based on IVUS data, if two intraluminal procedures (e.g., IVUS data and physiology data) are performed and coregistered to the same pathway (e.g., the pathway 1440), the same IVUS data and physiology data may be coregistered to each other, as shown by the iFR data 1492 shown at locations along the ILD 1450.

The length measurements along the ILD 1450 may be generated by a user of the system 100 and/or automatically by the processor circuit 510. For example, a user may select various locations along the ILD 1450 and the processor circuit may calculate length measurements corresponding to the selected locations. These various length measurements may also be displayed as metrics 1460 near the ILD 1450. In some embodiments, length measurements may be distinguished from one another by labels, colors, patterns, highlights, or other visual characteristics.

The indicators 1452 and 1456 may be user selected locations along the ILD 1450. In some embodiments, they may be automatically selected. As an example, the indicators 1452 and 1456 may identify the beginning and ending locations of a length measurement. In some embodiments, the indicators 1452 and 1456 correspond to a distal and proximal landing zone for a stent that is being considered by a physician. The iFR estimate value in the physiology data 1490 may be a predicted iFR value with proposed stent positioned within the vessel based on indicators 1452 and 1456. In some embodiments, corresponding indicators may be displayed at corresponding locations along the calculated footprint line 1440 of the image 1410.

In some embodiments, one or more bookmarks 1454 may also be included along the ILD 1450. These bookmarks 1454 may correspond to similar bookmarks at corresponding locations along the calculated footprint line 1440 of the image 1410.

An indicator 1470 is provided in the screen display 1400, overlaid on the x-ray image 1410. The indicator 1470 identifies for the user that the x-ray image is a zero contrast image frame.

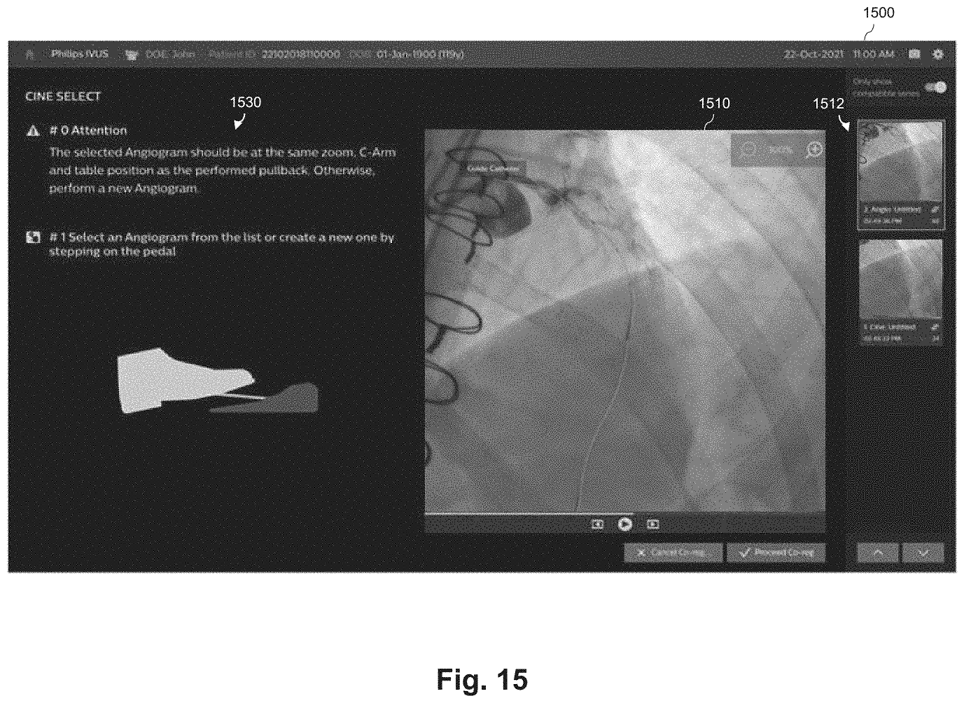

FIG. 15 is a diagrammatic view of a graphical user interface 1500, according to aspects of the present disclosure. As shown in FIG. 15, the graphical user interface may include an extraluminal image 1510, images 1512, and prompts 1530.

In some embodiments, the processor circuit 510 may initiate the steps of coregistering intraluminal data to an extraluminal image without contrast as has been described in response to a user input selecting an extraluminal image without contrast or by automatically detecting an extraluminal image without contrast. For example, as shown in FIG. 15, the processor circuit may display to a user multiple selectable options 1512 corresponding to an angiogram image (e.g., an x-ray image obtained with contrast) and a fluoroscopy image (e.g., an x-ray image obtained without contrast). In some embodiments, the selectable options 1512 may correspond to images. In some embodiments, the images 1512 may be exemplary images of an angiogram image obtained with contrast and a fluoroscopy or cine image obtained without contrast respectively. In some embodiments, these images may correspond to or be images of the specific patient's anatomy obtained during an imaging or treatment procedure. If a user selects an image corresponding to an image without contrast, the steps described in the present disclosure may be initiated by the processor circuit. If a user selects an image corresponding to an image with contrast, the steps of coregistering intraluminal data to a contrast-filled angiogram may be initiated by the processor circuit 510.

In an embodiment in which the processor circuit 510 automatically determines whether a contrast-filled angiogram or contrast-free fluoroscopy image or cine image is presented, the processor circuit 510 may receive an extraluminal image either from an extraluminal imaging system during a procedure or from a memory in communication with the processor circuit 510. In such an embodiment, the processor circuit 510 may employ any suitable image processing and/or machine learning techniques, including any of those listed in the present disclosure, to determine whether the received image is an angiogram image or a contrast-free image. If an angiogram was received, the steps of coregistration to an angiogram image may be commenced. If a contrast-free extraluminal image was received, the steps described herein may be initiated.

In some embodiments, the processor circuit 510 may be configured to display prompts, such as the prompts 1530 to guide a user at this stage of the procedure. For example, by displaying the prompts 1530, the processor circuit 510 may guide a user to select an existing angiogram image, fluoroscopy image, or cine image, and/or acquire an additional image by following the prompts 1530.

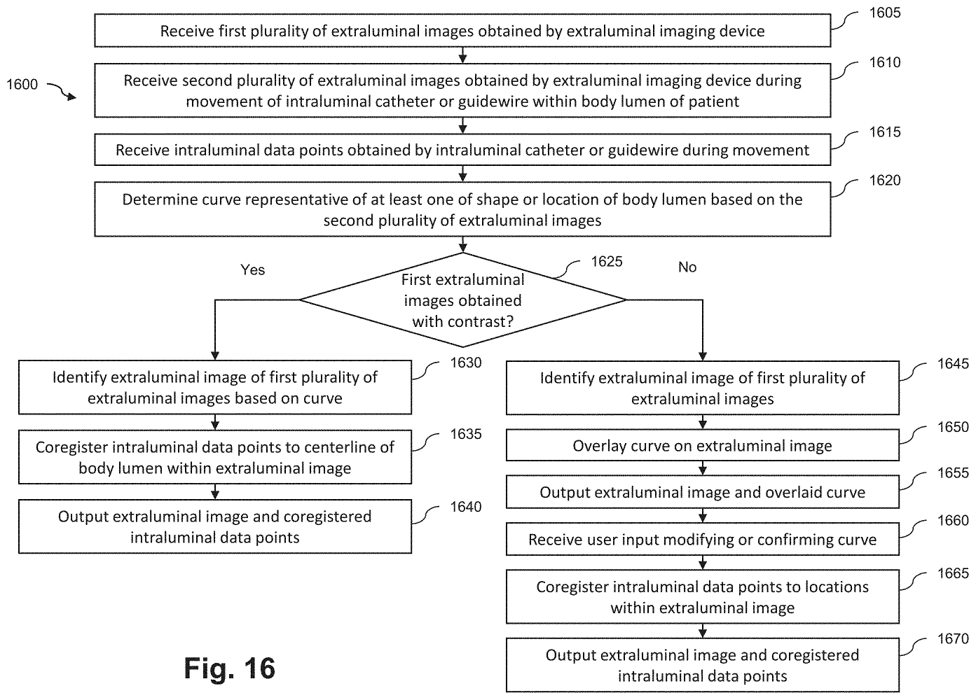

FIG. 16 is a flow diagram of a method of coregistering intraluminal data to a no contrast x-ray image frame, according to aspects of the present disclosure. The method 1600 may describe an automatic segmentation of a vessel to detect segments of interest using co-registration of invasive physiology and x-ray images. As illustrated, the method 1600 includes a number of enumerated steps, but embodiments of the method 1600 may include additional steps before, after, or in between the enumerated steps. In some embodiments, one or more of the enumerated steps may be omitted, performed in a different order, or performed concurrently. The steps of the method 1600 can be carried out by any suitable component within the system 100 and all steps need not be carried out by the same component. In some embodiments, one or more steps of the method 1600 can be performed by, or at the direction of, a processor circuit of the diagnostic system 100, including, e.g., the processor 560 (FIG. 5) or any other component.

At step 1605, the method 1600 includes receiving a first plurality of extraluminal images obtained by an extraluminal imaging device. The extraluminal imaging device may be a device of the extraluminal imaging system 151 shown and described with reference to FIG. 1. In some aspects, the extraluminal images of the first plurality of extraluminal images may be cine images. The extraluminal images may be acquired using increased radiation resulting in images of higher quality. In some aspects, the first plurality of extraluminal images may be angiographic frames. The first plurality of extraluminal images may be acquired with or without contrast.

At step 1610, the method 1600 includes receiving a second plurality of extraluminal images obtained by the extraluminal imaging device during movement of an intraluminal catheter or guidewire within a body lumen of the patient. In some aspects, the intraluminal catheter may be the intraluminal device 102 shown and described with reference to FIG. 1. In some aspects, the second plurality of extraluminal images may be fluoroscopic image frames. In some aspects, the second plurality of extraluminal images may be extraluminal images obtained with less radiation exposure than the first plurality of extraluminal images. The second plurality of extraluminal images may depict radiopaque portions of the intraluminal device as it moves through the body lumen of the patient. In procedures in which the heart, or other constantly and/or periodically moving organ or structure is imaged, the position of the radiopaque portions of the intraluminal device may be subject to the motion. This movement may exhibit a periodic or sinusoidal behavior. As a result, the path of the intraluminal device in the second plurality of extraluminal images may not match the centerline of the lumen imaged in a still image. In that regard, the second plurality of extraluminal images are obtained during a plurality of anatomical cycles such that the intraluminal catheter or guidewire experiences periodic motion during the movement of the intraluminal catheter or guidewire through the body lumen. This movement may include side-to-side movement, movement lateral to, perpendicular to, parallel to, or longitudinal with the movement of the intraluminal device. The second plurality of extraluminal images may be obtained during the same procedure or a separate procedure from the first plurality of extraluminal images.

At step 1615, the method 1600 includes receive intraluminal data points obtained by the intraluminal catheter or guidewire during movement. The intraluminal data points may be of any suitable type, including IVUS data, OCT data, intravascular pressure data, intravascular flow data, or any other data. In addition, the intraluminal data points are acquired simultaneously with the second plurality of extraluminal images.

At step 1620, the method 1600 includes determining a curve representative of at least one of a shape or a location of the body lumen based on the second plurality of extraluminal images. In some aspects, this curve may be referred to as a footprint line (FPL) and may be an approximation of the path of the intraluminal device through the body lumen if there was no motion in the patient anatomy. This calculated footprint line may be a coarse/smoothed representation of the body lumen or an average location of the body lumen. It is based, for example, on analysis of pullback images (e.g., the second plurality of extraluminal images) and the detection of the intraluminal device (e.g., the radio-opaque markers, such as a guidewire (GW) opaque tip or Guiding Catheter (GC)). In that regard, the calculated FPL is a coarse/smoothed representation of the vessel or an average location of the vessel (which is subject to periodic motion as described above). The curve may also be referred to as a line, a curve, a pathway, a centerline, a roadmap, or any other term.

At step 1625, the method 1600 includes determining whether the first plurality of extraluminal images were obtained with contrast. In some aspects, the processor circuit of the system 100 may analyze one or more extraluminal images of the first plurality of extraluminal images to determine whether they were obtained with or without contrast. For example, a machine learning algorithm, such as a neural network or any other deep learning network, may be implemented to automatically identify whether an extraluminal image was obtained with or without contrast. As shown in FIG. 16, if the processor circuit determines that the first plurality of extraluminal images were obtained with contrast agent, the processor circuit may perform steps 1630-1640, described below. If the processor circuit determines that the first plurality of extraluminal images were obtained without contrast agent, the processor circuit may perform steps 1645-1670, described after the description of steps 1630-1640.

At step 1630, the method 1600 includes identifying an extraluminal image of the first plurality of extraluminal images based on the curve. This extraluminal image may be selected automatically. For example, a processor circuit may extract the centerline of the imaged body lumen and compare it to the curve. In some aspects, the processor circuit may compare multiple positions of the curve with corresponding positions of the centerlines identified in each extraluminal image of the first plurality of extraluminal images. For example, for one extraluminal image, a proximal position of the centerline may be compared with the proximal position of the curve. This comparison may result in a distance between the two positions, for example, in units of pixels, or any other unit. This comparison may be performed for each point along the centerline and corresponding curve (e.g., the centerline and curve may be compared at a regular interval of distance, or the centerline and curve may be divided into an equal number of sections and comparisons may be made for each section). After position comparisons are performed, resulting in a number of distance values, these values may be averaged, summed, or otherwise combined to determine an overall comparison value for the extraluminal image analyzed. In that regard, the processor circuit may select an extraluminal image which has the ideal comparison value (e.g., lowest, closest to a reference value, highest, etc.) indicating that the shape of the body lumen within that extraluminal image aligns most closely with the curve generated at step 1620.

In some aspects, after the processor circuit selects an extraluminal image of the first plurality of extraluminal images, the user may verify that the selected extraluminal image ideally matches the curve. The user may then correct or select a new image. In some aspects, a user may manually correct the result of the automatically derived centerline and/or re-draw a new centerline altogether.

At step 1635, the method 1600 includes co-registering intraluminal data points to the centerline of body lumen within the extraluminal image. Because the intraluminal data points are associated with corresponding locations along the curve (e.g., the positions at which the intraluminal data points were acquired as observed in the second plurality of extraluminal images), the curve and its corresponding location information for the intraluminal data points may be overlaid on the selected extraluminal image. As a result, the locations at which intraluminal data points were acquired may be observed within the extraluminal image.

At step 1640, the method 1600 includes outputting the extraluminal image and coregistered intraluminal data points. This may include any suitable graphical user interface including the extraluminal image with an indication of a location at which an intraluminal data point was acquired along with the intraluminal data point.

As previously described, the processor circuit may alternatively perform steps 1645-1670 if it determines at step 1625 that the first plurality of extraluminal images were obtained without contrast. At step 1645, the method 1600 includes identifying an extraluminal image of the first plurality of extraluminal images. This extraluminal image may be selected based on the orientation of the extraluminal imaging device and patient. For example, the extraluminal image selected should be an image obtained from the same angle, and with the same imaging settings, as the second plurality of extraluminal images. In some aspects, the extraluminal image selected at step 1645 may alternatively be one of the second plurality of extraluminal images. Because the first plurality of extraluminal images were obtained without contrast, the body lumen and the centerline of the body lumen are not visible in the first extraluminal image. In some aspects, the extraluminal image identified at step 1645 may be an extraluminal image of the second plurality of extraluminal images received at step 1610.

At step 1650, the method 1600 includes overlaying the curve on the selected extraluminal image. In that regard, step 1650 includes setting the lumen centerline as the calculated FPL, or curve, in selected extraluminal image without contrast. In that regard, the processor circuit does not identify the extraluminal image at step 1645 based on the centerline of the body lumen as in step 1630. In some aspects, the processor circuit assigns the curve to be the centerline, without regard for what the actual location and shape of the body lumen and the centerline are. The processor circuit does this because the curve is a sufficiently accurate representation of the actual location and shape of the body lumen and the centerline.

At step 1655, the method 1600 includes outputting the extraluminal image and overlaid curve. This may include displaying the selected extraluminal image with the curve (e.g., calculated FPL) overlaid. A user may then review the curve within the extraluminal image and determine whether the curve accurately depicts the expected location of the body lumen based on observing the acquisition of the second plurality of extraluminal images.

At step 1660, the method 1600 includes receiving a user input modifying or confirming the curve. For example, if the user determines that a section of the curve should be modified, the user may use an input device, such as a touch screen, a mouse, a keyboard, various buttons of a graphical user interface, or any other means to adjust the curve as needed. In some aspects, the curve may not need to be modified. However, the user may provide a user input confirming that the shape of the curve looks accurate. In some aspects, the system workflow may make it mandatory for the user to review, correct and/or redraw altogether the vessel centerline.

At step 1665, the method 1600 includes coregister intraluminal data points to locations within the extraluminal image. For example, as described at step 1640, the intraluminal data points may be associated with various locations along the curve. These intraluminal data points may be similarly associated with corresponding locations within the extraluminal image selected.

At step 1670, the method 1600 includes outputting the extraluminal image and the coregistered intraluminal data points to a display. The step 1670 may be similar to the step 1640 previously described. For example, the display may provide any suitable graphical user interface including the extraluminal image with an indication of a location at which an intraluminal data point was acquired along with the intraluminal data point.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an extraluminal imaging device and an intraluminal catheter or guidewire, wherein the processor circuit is configured to:
receive a first extraluminal image obtained by the extraluminal imaging device;
receive a plurality of second extraluminal images obtained by the extraluminal imaging device during movement of the intraluminal catheter or guidewire within a body lumen of a patient, wherein the plurality of second plurality of extraluminal images are obtained without a contrast agent within the body lumen;
receive a plurality of intraluminal data points obtained by the intraluminal catheter or guidewire during the movement;
determine, based on the plurality of second extraluminal images, a curve representative of at least one of a shape or a location of the body lumen;
determine if the first extraluminal image was obtained without the contrast agent within the body lumen;
in response to the determination that the first extraluminal image was obtained without the contrast agent within the body lumen:
assign the curve to be a centerline of the body lumen in the first extraluminal image;
co-register the plurality of intraluminal data points to positions along the curve;
output, to a display in communication with the processor circuit, a first screen display comprising:
the first extraluminal image;
a visual representation of an intraluminal data point of the plurality of intraluminal data points; and
a marking overlaid on the extraluminal image at a corresponding position of the intraluminal data point.

2. The system of claim 1, wherein, in response to the determination that the first extraluminal image was obtained without the contrast agent within the body lumen, the processor circuit is configured to:
output, to the display, a second screen display comprising:
the first extraluminal image; and
the curve overlaid on the first extraluminal image.

3. The system of claim 2, wherein the second screen display comprises a plurality of user input options to at least one of accept the centerline, correct the centerline, or draw a new centerline.

4. The system of claim 3, wherein, when a user input option to correct the centerline is selected, the processor circuit is configured to receive a user input to identify a region of the curve and select a new location within the first extraluminal image corresponding to a corrected location of the region.

5. The system of claim 3, wherein the processor is configured to perform the co-registration and output the first screen display only after receiving a user input via the plurality of user input options.

6. The system of claim 1,
wherein the processor circuit is configured for communication with a touchscreen display,
wherein the processor circuit is configured to output the first screen display to the touchscreen display, and
wherein the processor circuit is configured to receive the user input from the touchscreen display.

7. The system of claim 1, wherein the extraluminal imaging device comprises an x-ray imaging device.

8. The system of claim 7, wherein the first extraluminal image is obtained with a first radiation dose and the plurality of second extraluminal images are obtained with a second radiation dose smaller than the first radiation dose.

9. The system of claim 1, wherein the processor circuit is configured to:
receive a plurality of first extraluminal images obtained by the extraluminal imaging device; and
select the first extraluminal image from among the plurality of first extraluminal images.

10. The system of claim 1, wherein the processor circuit is configured to determine if the first extraluminal image was obtained without the contrast agent automatically, without receiving a user input to identify that the first extraluminal image was obtained without the contrast agent.

11. The system of claim 1,
wherein the plurality of second extraluminal images show a radiopaque portion of the intraluminal catheter or guidewire, and
wherein the processor circuit is configured to determine the curve based on the radiopaque portion shown in the plurality of second extraluminal images.

12. The system of claim 11,
wherein the plurality of second extraluminal images are obtained during a plurality of anatomical cycles such that the intraluminal catheter or guidewire experiences periodic motion during the movement of the intraluminal catheter or guidewire through the body lumen, and
wherein, to determine the curve, the processor circuit is configured to perform motion compensation.

13. The system of claim 12, wherein, to perform the motion compensation, the processor circuit is further configured to locate the curve along a center of a shape generated by the movement of the intraluminal catheter or guidewire within the body lumen while the intraluminal catheter or guidewire experiences the periodic motion.

14. The system of claim 1, wherein the first extraluminal image is one of the plurality of second extraluminal images.

15. The system of claim 1, wherein the processor circuit is further configured to assign the curve to be a centerline of the body lumen in the first extraluminal image without identifying the body lumen in the first extraluminal image and without identifying the centerline in the first extraluminal image.

16. A system, comprising:
a processor circuit configured for communication with an extraluminal imaging device and an intraluminal catheter or guidewire, wherein the processor circuit is configured to:
receive a first extraluminal image obtained by the extraluminal imaging device, wherein the first extraluminal image is obtained without contrast agent within the body lumen;
receive a plurality of second extraluminal images obtained by the extraluminal imaging device during movement of the intraluminal catheter or guidewire within a body lumen of a patient, wherein the plurality of second plurality of extraluminal images are obtained without a contrast agent within the body lumen;
receive a plurality of intraluminal data points obtained by the intraluminal catheter or guidewire during the movement;
co-register the plurality of intraluminal data points to the first extraluminal image based on the plurality of second extraluminal images such that the co-registration is performed without an extraluminal image obtained with contrast agent within the body lumen;
output, to a display in communication with the processor circuit, a first screen display comprising:
the first extraluminal image;
a visual representation of an intraluminal data point of the plurality of intraluminal data points; and
a marking overlaid on the extraluminal image at a corresponding position of the intraluminal data point.

17. A system, comprising:
an intravascular imaging catheter; and
a processor circuit configured for communication with an x-ray imaging device and the intravascular imaging device, wherein the processor circuit is configured to:
receive a first x-ray image obtained by the x-ray imaging device;
receive a plurality of second x-ray images obtained by the x-ray imaging device during movement of the intravascular imaging catheter within a blood vessel of a patient, wherein the plurality of second plurality of x-ray images are obtained without a contrast agent within the blood vessel;
receive a plurality of intravascular images obtained by the intravascular imaging catheter during the movement;
determine, based on the plurality of second x-ray images, a curve representative of at least one of a shape or a location of the blood vessel;
determine if the first x-ray image was obtained without the contrast agent within the blood vessel;
in response to the determination that the first x-ray image was obtained without the contrast agent within the blood vessel:
assign the curve to be a centerline of the body lumen in the first x-ray image without identifying the blood vessel in the first x-ray image and without identifying the centerline in the first x-ray image;
co-register the plurality of intravascular images to positions along the curve;
output, to a display in communication with the processor circuit, a first screen display comprising:
the first x-ray image;
an intravascular image of the plurality of intravascular images; and
a marking overlaid on the extraluminal image at a corresponding position of the intravascular image.
